(19) United States
(12) Patent Application Publication
(10) Pub. No.: US 2025/0143899 A1
(43) Pub. Date: May 8, 2025

(54) VASCULAR FLOW MANAGEMENT

(71) Applicant: EDWARDS LIFESCIENCES CORPORATION, Irvine, CA (US)

(72) Inventors: Michael G. Valdez, Riverside, CA (US); Emil Karapetian, Huntington Beach, CA (US); Haim Brauon, Beit Dagan (IL)

(21) Appl. No.: 19/015,032

(22) Filed: Jan. 9, 2025

Related U.S. Application Data
(63) Continuation of application No. PCT/US2023/070984, filed on Jul. 25, 2023.
(60) Provisional application No. 63/370,158, filed on Aug. 2, 2022.

(51) Int. Cl. A61F 2/90; A61F 2/958

(52) U.S. Cl. CPC A61F 2/90; A61F 2/958; A61F 2220/0016; A61F 2220/0075; A61F 2230/0069; A61F 2250/0039

(57) ABSTRACT

Implant devices are provided for restoring compliance characteristics to blood vessels. An exemplary implant device includes an outer tubular frame having a diameter than varies along a longitudinal axis and a radially expandable tube disposed within the frame. Further disclosed are implant devices including a frame having an axially convex form that expands in diameter moving from first and second axial ends of the frame to a medial portion of the frame, and a cylindrical toroidal balloon member comprising one or more gas-filled chambers disposed at least partially within the frame. Further disclosed are implant devices including a tubular frame comprising a first axial segment and a second axial segment, a radially expandable tube disposed within the first axial segment of the frame, and a one-way valve disposed within the second axial segment of the frame.

### RELATED APPLICATION(S)

This application is a continuation of International Patent Application No. PCT/US23/70984, filed Jul. 25, 2023, which claims the benefit of U.S. Provisional Patent Application Ser. No. 63/370,158, filed on Aug. 2, 2022, the complete disclosures of which are hereby incorporated by reference in their entirety.

### BACKGROUND

### Field

The present disclosure generally relates to the field of medical implant devices.

### Description of Related Art

Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and systems that facilitate the restoration of compliance characteristics to undesirably stiff blood vessels. Devices associated with the various examples of the present disclosure can include a stent frame configured to dilate a native blood vessel and provide an internal space within which a fluid channel/tube may expand and retract/recoil to thereby provide a change in volume of the channel over the cardiac cycle. Such change in volume can allow the blood vessel to mimic compliance of a healthy blood vessel and/or otherwise promote blood flow during, for example, the diastolic phase of the cardiac cycle.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIGS. 1A and 1B illustrate example representations of cardiac and vascular anatomy of a patient.

FIGS. 2A and 2B provide cross-sectional and side views, respectively, of a blood vessel experiencing compliant contraction during the diastolic phase of the cardiac cycle.

FIGS. 3A and 3B provide cross-sectional and side views, respectively, of the artery shown in FIGS. 2A and 2B during the systolic phase of the cardiac cycle during which the blood vessel experiences compliant expansion.

FIG. 4 is a graph illustrating blood pressure over time in an example healthy patient.

FIG. 5 is a graph illustrating blood pressure over time in an example patient having reduced aortic compliance.

FIGS. 6A-6E provide views of a compliance-enhancing implant device including one or more compressible fluid chambers in accordance with one or more examples.

FIGS. 7A and 7B show side cross-sectional views of the compliance-enhancing implant device of FIGS. 6A-6E in compressed and relaxed configurations, respectively, in accordance with one or more examples,

FIGS. 8A-8E provide views of a compliance-enhancing implant device including an inner elastic tube in accordance with one or more examples.

FIGS. 9A and 9B shows side cross-sectional views of the compliance-enhancing implant device of FIGS. 8A-8E in expanded/stretched and relaxed configurations, respectively, in accordance with one or more examples.

FIGS. 10A, 10B, and 10C show cross-sectional side views of compliance implant devices having respective flow channel/tube shapes/configurations in accordance with various examples.

FIG. 11 shows a compliance-enhancing implant device including an internal elastic tube that is sutured at proximal and/or distal ends thereof to an outer frame in accordance with one or more examples.

FIG. 12 shows a compliance-enhancing implant device including an internal elastic tube that has one or more circumferential inlets/outlets and/or fluid-flow channels/pleats in accordance with one or more examples,

FIGS. 13A and 13B show side views of a compliance-enhancing implant device in relaxed and expanded configurations, respectively, in accordance with one or more examples.

FIGS. 14A, 14B, and 14C illustrate a flow diagram for a process for implanting a compliance-enhancing implant device in accordance with one or more examples.

FIGS. 15A, 15B, and 15C provide images of the compliance-enhancing implant device and certain anatomy corresponding to operations of the process of FIGS. 14A, 14B, and 14C according to one or more examples.

FIGS. 16A and 16B show compliance-enhancement action of an implant device during high-pressure and low-pressure phases/conditions, respectively.

FIGS. 17A, 17B, and 17C provide side, side cross-sectional, and axial views, respectively, of a valved compliance-enhancing implant device in accordance with one or more examples.

FIG. 18A shows a compliance-enhancing implant device including a valve portion in accordance with one or more examples.

FIGS. 18B, 18C, and 18D show respective designs of valve portions of compliance-enhancing implant devices in accordance with one or more examples.

FIG. 19 shows aortic anatomy with valved compliance-enhancing implant devices deployed in example positions in the anatomy in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described, Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, compliance-enhancement implant devices implanted in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

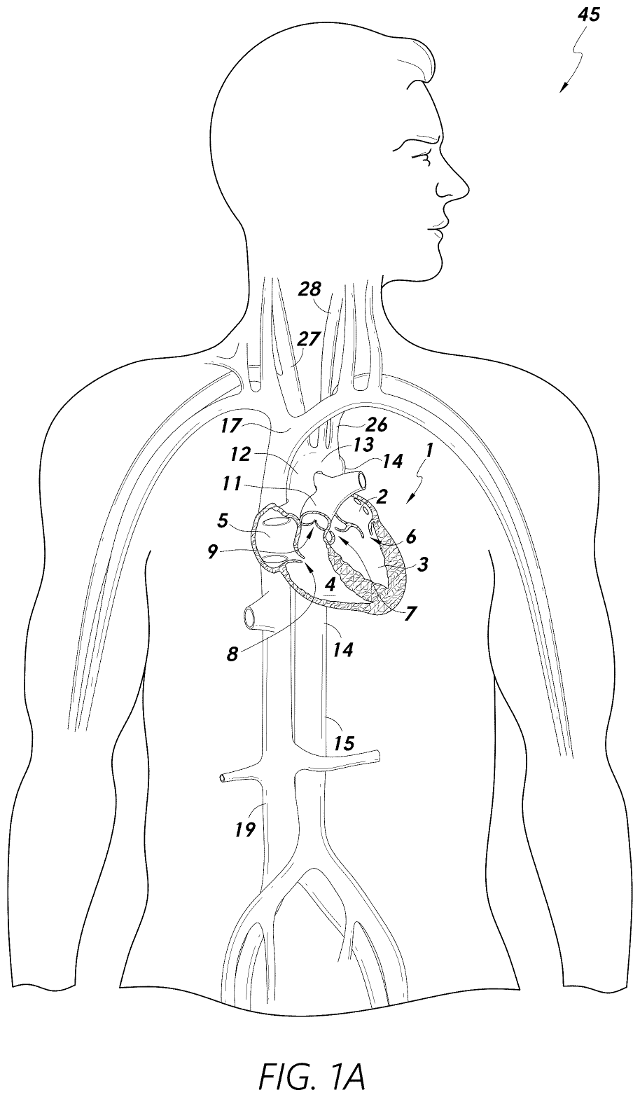

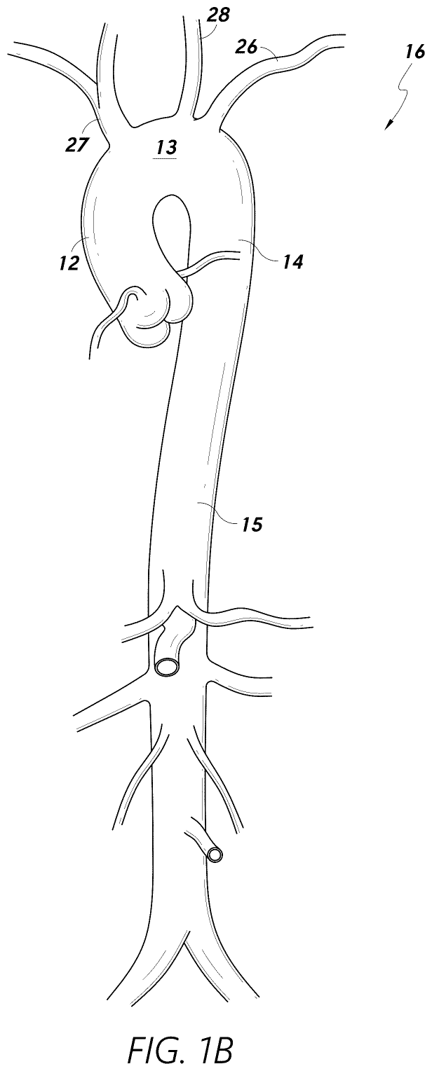

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5, In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole), The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery, and the right aortic sinus likewise gives rise to the right coronary artery. Together, these two arteries supply the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

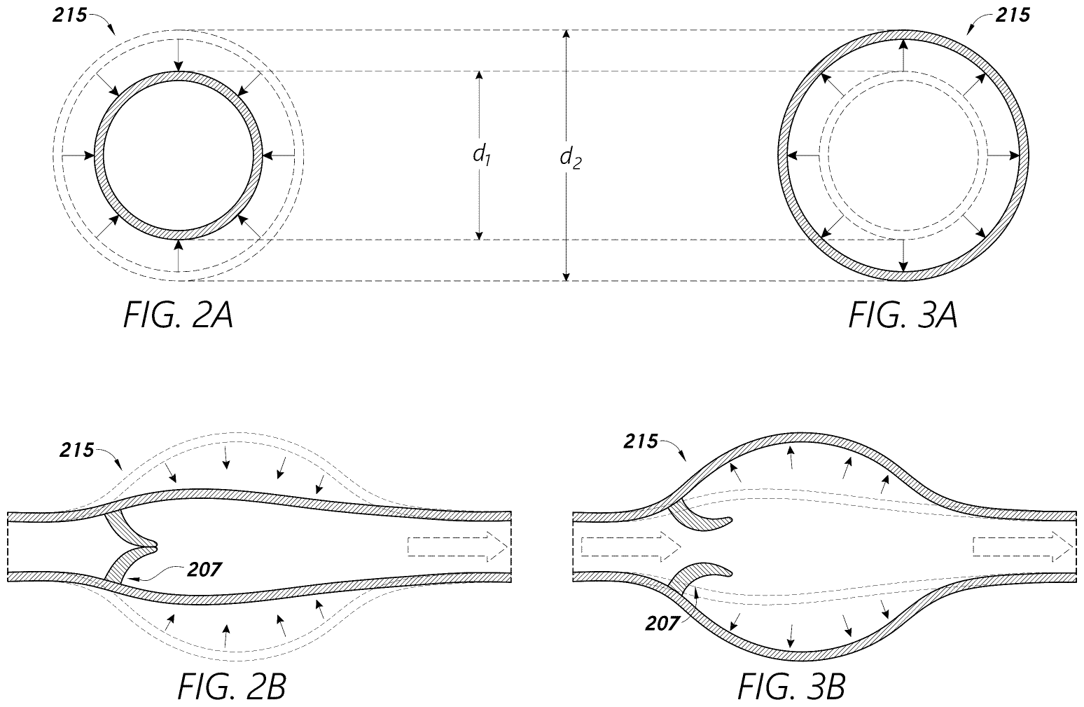

FIGS. 2A and 2B provide cross-sectional and side views, respectively, of a compliant blood vessel 215, such as an artery (e.g., aorta), experiencing compliant radial contraction during the diastolic phase of the cardiac cycle, whereas FIGS. 3A and 3B show the blood vessel 215 experiencing compliant expansion during the systolic phase of the cardiac cycle. As understood by those having ordinary skill in the art, the systolic phase of the cardiac cycle is associated with the pumping phase of the left ventricle, while the diastolic phase of the cardiac cycle is associated with the resting or filling phase of the left ventricle. As shown in FIGS. 3A and 3B, with proper arterial compliance, an increase in volume will generally occur in an artery when the pressure in the artery is increased. With respect to the aorta, as shown in FIGS. 3A and 3B, as blood is pumped into the aorta 215 through the aortic valve 207, the pressure in the aorta increases and the diameter of at least a portion of the aorta expands. A first portion of the blood entering the aorta 215 during systole may pass through the aorta during the systolic phase, while a second portion (e.g., approximately half of the total blood volume) may be stored in the expanded volume caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

As shown in FIGS. 2A and 2B, arterial compliance may cause retraction/recoil of the blood vessel wall inward during diastole, thereby creating pressure in the blood vessel to cause blood to continue to be pushed through the artery 215 when the valve 207 is closed. For example, during systole, approximately 50% of the blood that enters the artery 215 through the valve 207 may be passed through the artery, whereas the remaining 50% may be stored in the artery, as enabled by expansion of the vessel wall. Some or all of the stored portion of blood in the artery 215 may be pushed through the artery by the contracting vessel wall during diastole. For patients experiencing arterial stiffness that causes lack of compliance, their arteries may not operate effectively in accordance with the expansion/contraction functionality shown in FIGS. 2A and 2B and FIGS. 3A and 3B.

Arterial compliance restoration devices, methods, and concepts disclosed herein may be generally described in the context of the ascending aorta. However, it should be understood that such devices, methods and/or concepts may be applicable in connection with any other artery or blood vessel.

FIG. 4 is a graph illustrating blood pressure over time in an example patient with a healthy, compliant aorta, wherein arterial blood pressure is represented as a combination of a forward systolic pressure wave 702 and a backward diastolic pressure wave 701, The combination of the systolic wave 702 and the diastolic wave 701 are represented by the waveform 703.

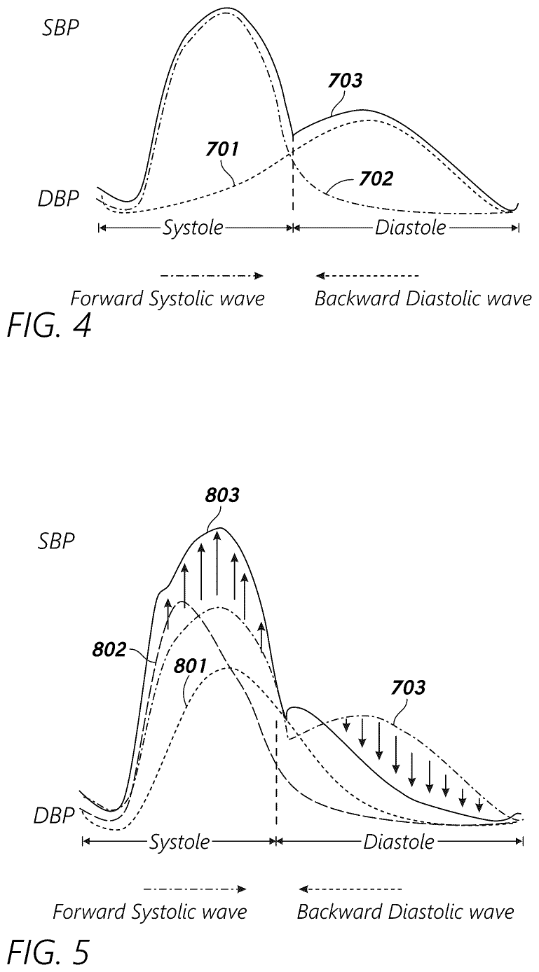

FIG. 5 is a graph illustrating blood pressure over time in an example patient having reduced aortic compliance. The graph of FIG. 5 shows, for reference purposes, the example combined wave 703 shown in FIG. 4. When low compliance is exhibited, less energy may be stored in the aorta compared to a healthy patient. Therefore, the systolic waveform 802 may demonstrate increased pressure during the systolic phase relative to a patient having normal compliance, while the diastolic waveform 801 may demonstrate reduced pressure during the diastolic phase relative to a patient having normal compliance. Therefore, the resulting combined waveform 803 may represent an increase in the systolic peak and a drop in the diastolic pressure, which may cause various health complications. For example, the change in waveform may impact the workload on the left ventricle and may adversely affect coronary profusion.

In view of the health complications that may be associated with reduced arterial compliance, as described above, it may be desirable in certain patients and/or under certain conditions, to at least partially alter compliance properties of the aorta or other artery or blood vessel, or otherwise alter/control flow therein, in order to improve cardiac and/or other organ health. Disclosed herein are various devices and methods for at least partially restoring compliance to a blood vessel, such as the aorta. Certain examples disclosed herein achieve restoration of arterial compliance through the use of implantable compliant fluid channels/tubes, through which blood circulation may flow. For example, a compliance-restoration device in accordance with the present disclosure may comprise an expandable fluid channel that expands and stores energy during higher-pressure periods of the cardiac cycle (e.g., during the systolic phase) and contracts/compresses during lower-pressure period (e.g., during the diastolic phase) to return the stored energy to the circulation and increase flow through the channel.

In some examples, devices of the present disclosure include compressible fluid (e.g., gas) chamber(s) surrounding the compliant fluid channel of the device, wherein the chamber(s) may be disposed within a vessel-dilating outer frame. In some examples, devices of the present disclosure include elastic/compliant tubes/channels disposed within a blood-vessel-dilating frame, which provides a space outside of the compliant tube/channel that can contain circulatory blood, which may cyclically fill and empty from the space in a manner as to increase compliance and/or diastolic flow in the blood vessel. The compliant channel/tube can be secured in-place to the frame (e.g., stent frame), which may comprise metal or other at least partially rigid material. Such frame can be configured to expand within the target blood vessel to cause dilation thereof, wherein the dilation of the blood vessel can serve to both secure the frame in the desired position within the target blood vessel, and further to create a space within the blood vessel in which the compliant channel/tube can radially expand.

Devices of the present disclosure may include additional anchoring features to provide secure retention in the target blood vessel. For example, barb-type anchors may be integrated with the outer frame. Certain coverings and/or linings (e.g., cloth, polymer) may be implemented on the outer frame to improve fluid-sealing characteristics of the implant device and/or promote in-growth with the native blood vessel tissue. Compliance restoration devices disclosed herein may serve to at least partially increase coronary perfusion.

### Compliance-Enhancing Implant Devices

The present disclosure relates to systems, devices, and methods for adding back and/or increasing compliance to the aorta or other arterial (or venous) blood vessel(s) to provide improved perfusion of the heart muscle and/or other organ(s) of the body. Examples of the present disclosure can include compliant tubular devices configured to channel blood circulation therethrough, such that compliant/elastic expansion of the tube during systole can be returned to the circulation during diastole to thereby reduce systolic pressure and increase diastolic pressure. By disposing compliant tubular implants within an at least partially rigid outer frame that is expanded and maintained within the native blood vessel, as opposed to solutions involving blood vessel grafts and/or resection, incidences of blood leakage and/or rupture of the expandable inner tube can be contained within the target blood vessel, thereby reducing hazards associated with extravascular arterial blood leakage, such as within the abdominal and/or chest cavity.

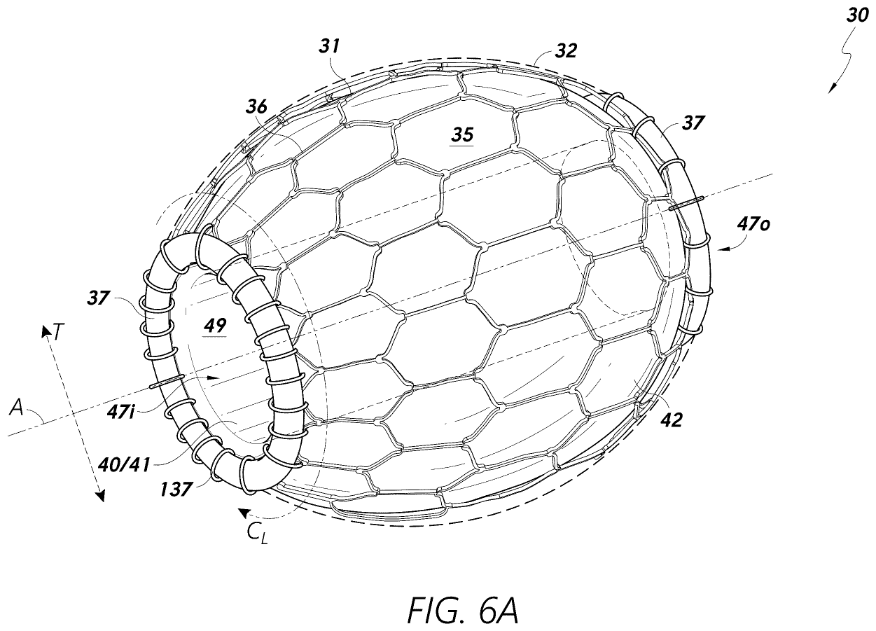

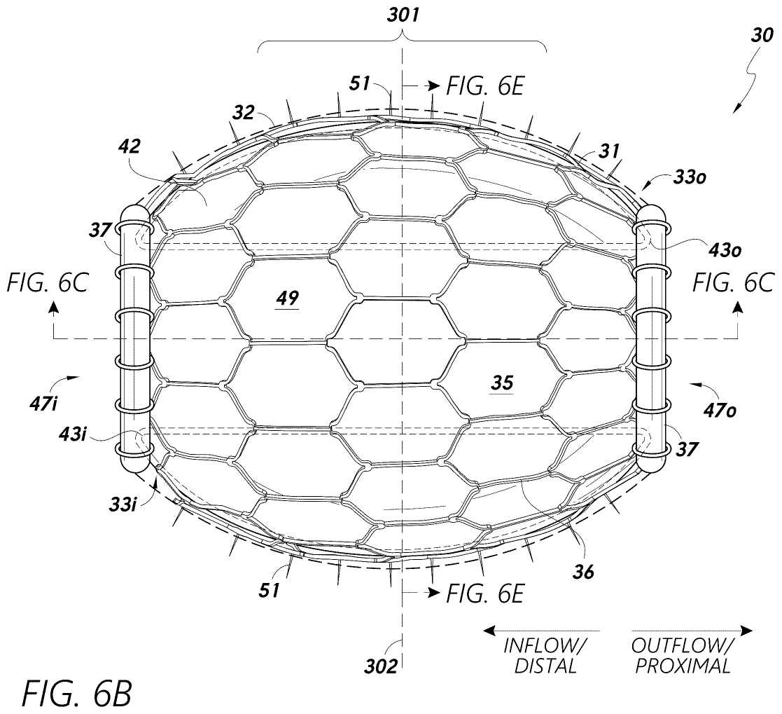

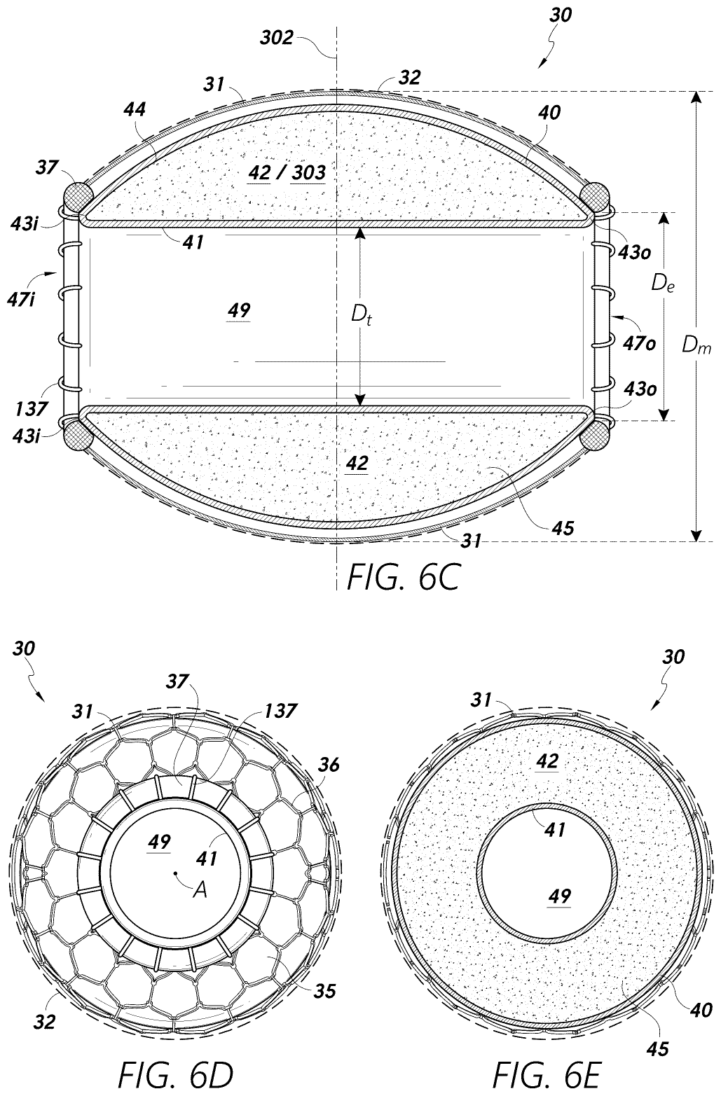

FIGS. 6A-6E provide views of a compliance implant device 30 including a tubular structure 40 having an axial internal/central fluid channel 49 formed by an inner tube/layer 41 that is configured to radially expand and retract/recoil/compress based on changing pressure conditions within the channel 49. The radially-expandable tube 41 may have disposed thereabout one or more sealed, compressible fluid chambers 42, wherein radial expansion, which causes an increase in the volume of the channel 49, causes compression of the chamber(s) 42 and/or fluid/medium (e.g., gas and/or foam) 45 therein. As the inner tube 41 radially expands and contracts, the volume of the channel 49 increases and decreases in a manner as to provide compliance characteristics for the blood vessel in which it is implanted. The compliant channel 49 is defined/formed by the expandable inner tube 41. The inner tube 41 may further serve as a radially-internal diameter/boundary of the compressible fluid chamber(s) 42.

The frame 31 is tubular in form with an axial channel therethrough, and is configured to expand to an expanded medial diameter Dₘ (e.g., in the medial area 301 thereof), wherein such expansion of the frame 31 within the native blood vessel (e.g., aorta) may serve to dilate the native blood vessel to some degree. For example, the medial diameter Dₘ of the frame may advantageously be at least 20% greater than the diameter of the native blood vessel in the area in which the frame is deployed. In some implementations, the diameter Dₘ is at least 30%, 35%, 40%, 45%, or 50% greater than the diameter of the native blood vessel. Therefore, it should be understood that examples of the present disclosure relate to compliance implant devices that perform a blood vessel dilation function when implanted/deployed, thereby substantially increasing the diameter of the native blood vessel and maintaining such dilation when the implant is disposed/implanted at the target anatomical site, wherein such dilation can serve to provide a space for radial expansion of the inner tube/layer 41. For example, the compressible gas 45 in the chamber(s) 42 can be compressed in the space provided between the inner tube 41 and the dilated/expanded blood vessel and frame 31.

Dilation of the native blood vessel, as relating to examples of the present disclosure, can present certain risks to the patient. For example, particularly with respect to blood vessel segments that are relatively stiff and/or thin, dilation of the blood vessel can result in rupture or other tissue damage. Where such rupture causes hemorrhaging, such event can severely impact the patient's health and can lead to severe injury or death. Concepts of the present disclosure can be implemented in a manner as to reduce such risks. For example, some implementations, such as the example shown in FIGS. 6A-6E, can include outer frames 31 that have gradually-increasing diameter moving from the axial ends 33 of the frame 31 towards the axial center 302, as shown, which can provide reduced strain on the native blood vessel tissue compared to examples in which the diameter of the frame is both greater than the native blood vessel and constant along the entire length thereof. Furthermore, it may be desirable for expansion/deployment of the frame 31 to be implemented in a gradual and nontraumatic manner. For example, self-expansion or balloon-expansion of the frame may be implemented relatively slowly to allow the native tissue to acclimate and respond gradually to the dilation from its natural diameter to the expanded diameter Dₘ.

The implant device 30 is configured to add back compliance to a target blood vessel in which it is implanted, such as the aorta, to improve perfusion of the heart muscle. The device 30 may be a percutaneously-placeable implant configured to be compressed (e.g., radially compressed) and transported within a delivery catheter or other tubular delivery system. The radially-expandable inner tube 41, in a natural, relaxed, and/or de-pressurized configuration/state can have a straight cylindrical shape/form, whereas in a radially-expanded, pressurized configuration/state, the tube 41 may have an outwardly-/externally-convex (e.g., internally concave) cylindrical shape, which may be similar to the shape of the expanded frame 31 (described in detail below) of the device 30. The device 30 may advantageously function as an arterial flow optimizer to generate vascular compliance.

The device 30 includes the outer frame 31, which may be an expandable stent-type frame configured to expand radially from a compressed delivery configuration to the expanded state shown in FIG. 6A. To achieve this change in the shape and dimension of the frame/stent 31, the frame 31 may have a structure comprising a plurality of struts forming an array of cells 35, which may have any suitable or desirable shape (e.g., oval/ellipse, diamond/rhombus, hexagonal diamond/polygon, etc.). The cells 35 may be arranged in any number of columns in the circumferential direction and rows in the axial, or lengthwise, direction A. In some examples, a short axis of the cells 35 extends in the circumferential direction and a long axis of the cells 35 extends in the axial direction A, or vice versa. In some examples, adjacent cells 35 are connected to each other in the circumferential and/or axial direction by connecting struts 36.

The cells 35 of the frame 31 may be formed using any suitable process, such as by stamping or machining the frame structure from a sheet or tube of metal. The frame 31 may be made of any at least partially rigid material, such as metal or plastic. For example, the frame may comprise stainless steel or nitinol. Where nitinol or other shape-memory metal or material is implemented for the frame 31, the frame may be self-expanding. In some implementations, the array of struts 36 is formed from a sheet of metal, which is rolled into a cylinder to form the tubular/cylindrical form of the frame configured for placement within a blood vessel. In some implementations, a balloon catheter can be used to expand the frame 31 for securing in the wall of an artery or other blood vessel or body cavity.

As referenced above, in addition to the frame 31, the device 31 includes an expandable (e.g., elastic) tube/channel 41 disposed within the frame 31. In some implementations, as in the example of FIGS. 6A-6E, the tube 41 is part of an elastic, multilayer cylindrical toroid (e.g., axially-stretched-donut-shaped) balloon inner member 40, wherein the inner member 40 is configured such that outer 44 and inner 41 layers/walls of the inner member/tube 40 are compressed/brought-together in response to a pressure gradient between the blood flowing through the axial channel 49 of the inner member/tube 40 and the gas or other fluid/medium 45 disposed within the compartment(s)/chamber(s) 42 of the inner cylindrical balloon 40. The device 30 may be implanted in, for example, the ascending aorta, such as in an area just above the aortic valve.

The radially-expandable inner tube/layer 41 of the compliance tube/member 40, which is disposed within the cylindrical outer stent frame 31, forms an axial blood flow channel/conduit 49 therethrough. In some examples, the outside of the frame 31 is covered with a fabric or polymer cover 32. The stent frame 31 may be a self-expandable and/or balloon-expandable stent. The frame 31 and/or balloon tube 40 can have an axially bulbous/bulging outer diameter, such that the diameter thereof is greater in the axial center Dₘ than at the axial ends Dₑ, as shown. Such bulbous/bulging shape can facilitate tighter fit of the device in the target blood vessel in which it is implanted.

The frame 31 and/or outer layer 44 of the internal balloon/tube 40 may advantageously have a shape that accommodates and facilitates compliant expansion of the inner tubular layer 41. For example, the inner tube 41 may define the flow channel 49 as having a diameter Dₜ in a relaxed/non-compressed (e.g., diastolic) configuration/state. For example, the diameter Dₜ of the tube 41 may be approximately equal to the diameter Dₑ of the end portions of the frame 31, such as within 10% of the dimension/distance of the diameter Dₑ. As possible example values that can be deviated from while still remaining within the context of the present disclosure, Dₜ may be a value/dimension between approximately 1-2 cm, D_{M} may be a value/dimension between 3-6 cm, and Dₑ may be a value/dimension between 1.5-3 cm in some implementations.

As shown, the frame 31 may have a shape of a convex cylinder, wherein a diameter of the frame 31 is nonuniform along an axis/length L_{f} of the frame 31. For example, as shown, the diameter Dₑ of the frame may be relatively smaller/narrower at one or both ends 33i, 330 of the frame, wherein the diameter of the frame may increase moving from the ends 33 towards an axial/lengthwise center 302 of the frame 31 to the expanded diameter Dₘ. In some examples, the increase in diameter moving towards the axial center 302 may be gradual and/or continuous, as in the illustrated example. However, in some examples, the diameter of the frame may expand in a stepwise manner, such that the maximum diameter Dₘ covers a substantial portion of the length of the frame (e.g., more than half of the length of the frame). For example, the frame 31 may increase in diameter from the narrower frame diameter Dₑ to the expanded frame diameter Dₘ in the end/outer portions/quartiles of the length L_{f} of the frame 31, such that the expanded diameter spans a medial section 301 of the frame that represents at least half of the total length L_{f} of the frame 31.

In some applications, the diameter Dₑ of the end portions 33 of the frame 31 may be sized to match the diameter of the native blood vessel in which the frame 31 is deployed. For example, it may be desirable for the diameter Dₑ of the end portions 33 of the frame 31 to be within 20% of the diameter of the native aorta in the particular target segment thereof. Generally, the diameter of the ascending aorta may be less than about 2.1 cm. With regard to the abdominal aorta, the diameter may be less than about 3.0 cm. The wall of the aorta may consist of three layers, including the intima, media, and adventitia layers. The intima is relatively thin, whereas the media contains the elastic fibers and smooth muscle cells forming a spiral layer of tissue providing the strength of the aortic wall, and the adventitia provides the nutrition with the arterial and venous vasa vasorum. The total wall thickness of the aorta in areas in which implants in accordance with the present disclosure may be implanted may generally be less than about 4 mm. The frame 31 is advantageously configured to dilate a target aortic vessel to a diameter at an axial center of the frame that is greater than 20% wider than the respective aortic diameters recited above, depending on the particular target blood vessel segment in which the implant is implanted, while not causing rupturing or undue trauma to the native vessel wall.

The convex cylindrical shape of the frame 31, when combined with the generally straight cylindrical shape/form of the inner tube/layer/channel 41, may advantageously provide a space 303 between the frame 31 and the tubular channel 49 defined by the inner tube/layer 41 when the tube/layer 41 is in a relaxed configuration, wherein the tube/layer 41 can expand within the space 303 to provide compliance. The inner tube/layer 41 may be configured to cycle between a relaxed state in low-pressure periods (e.g., diastolic phase of the cardiac cycle), wherein the tube 41 is generally cylindrical or slightly concave or convex in the relaxed state, and a radially expanded state in high-pressure periods (e.g., systolic phase of the cardiac cycle), wherein the tube 41 radially expands/bows-outward towards the frame 31 within the space 303 provided by the dilation of the blood vessel by the frame 31. That is, the shape and dimensions of the frame 31 advantageously provides a space 303 within which the tube/layer 41 is permitted to radially expand, wherein such expansion may take place within the dilated target blood vessel. By allowing for compliant expansion that is to a diameter that is greater than the natural, pre-implantation diameter of the native blood vessel (e.g., at least 20% greater than the diameter of the native blood vessel prior to expansion thereof by the frame 31), while providing for such expansion within the native blood vessel without requiring grafting, cutting, and/or resection of the native blood vessel (e.g., aorta), risks associated with blood vessel rupture can be reduced or eliminated. In some examples, the space 303 is primarily occupied by the fluid chamber(s) 42 between the inner tube 41 and the outer layer 44 of the compressible balloon 40, wherein radial expansion of the inner tube layer 41 causes compression of the fluid (e.g., gas) 45 disposed within the chamber(s) 42 of the balloon 40, such that the space 303 between the inner tube layer 41 and the frame 31 and outer layer 44 is reduced as the pressure in the channel 49 increases and causes the tube 41 to expand.

Without dilation of the native blood vessel, as effected by the frame 31, the space available within the frame for blood flow and expansion of the inner tube/layer 41 would be limited. For example, if the frame 31 had a constant diameter that was only slightly larger than the diameter Dₜ of the tubular channel 49, the inner tube/layer 41 would be unable to radially expand by a significant amount due to interference of the frame. Therefore, the ability of the tube 41 to change in volume cyclically in a manner as to add compliance to the blood circulation would be limited. That is, the convex cylindrical shape of the frame 31, wherein the frame has, at its maximum diameter, a diameter that is at least 20%, 30%, 40%, and/or 50% greater than the diameter of the native blood vessel, increases the ability of the inner tube 41 to add compliance to the blood circulation.

The outer frame 31 may be at least partially covered with a fabric or polymer coating or cover 32, which may promote tissue ingrowth with the inner diameter of the native blood vessel. The cover 32 may be disposed on an outer surface or area of the frame 31, and/or may be disposed/applied to the inner diameter of the frame 31 on an inside thereof. For example, the layer(s) of covering 32 may be disposed in some implementations between the outer frame 31 and the tube 40. In some implementations, the cover 32 comprises a cloth or polymer sleeve which may be at least partially elastic, or alternatively nonelastic. The covering 32 may be applied over or within the frame 31 in any suitable or desirable manner. For example, in some implementations, the cover 31 may be applied using an electrical or mechanical spinning (e.g., rotary jet spinning, electrospinning, or similar) application process or other deposition process known to those having ordinary skill in the art.

The frame 31 may be a self-expandable and/or balloon-expandable stent frame. As described above, the frame 31 and/or tubular member 40 may have an axially bulbous/bulging outer diameter, such that the diameter thereof is substantially greater in the axial center than at one or more of the axial ends 33 thereof, as shown. Such bulbous/bulging shape can facilitate tighter fit of the device 30 in the blood vessel in which it is implanted. The frame 31 can include barbs or other anchors 51 associated with an outer diameter thereof to engage/embed-in the target native blood vessel wall and secure the device in place. In some implementations, the barbs/anchors 51 puncture through an outer cover/coating 32 and into the native tissue to anchor the frame 31 in place.

The compliance-enhancement tube 40, which may include one or more compressible fluid/gas chambers 42 as shown, can be coupled to the frame 31 at one or both axial ends 33 of the frame 31. For example, the tubular balloon 40 can be sutured to respective ring structures 37 at either or both axial ends 33 of the stent frame 31. For example, such ring structures 37 may be integrated with the frame 31 or may be attached to one or both axial ends 33 thereof in some manner. For example, sutures, wires, or other coupling means 137 may be used to attach the ring(s) 37 to the frame 31.

The compressible tube 40 may further be sutured or otherwise secured/attached to the axial end(s) 33 of the frame 31. For example, the tube 40 may be sutured to the suture ring(s) 37 or other structure of the frame 31, or may be attached using an adhesive or other attachment means. In some implementations, distal end portion(s) 43 of the tube 40 may be heat-sealed, machine-crimped, or otherwise sealed in a manner such that suturing through, and/or apertures formed in, such portions may not provide fluid access to the internal chamber(s) 42 or otherwise break the seal thereof. That is, distal end portion(s) 43i, 430 of the tube 40 may comprise one or more layers of material with no fluid-filled chamber(s) disposed, formed, or exposed therein to allow for suturing or other attachment without the risk of puncture of the chamber(s) 42. In some implementations, axial end portions 433 of the tube 40 may be folded/wrapped around end portions 33 of the frame 31 and secured to the frame on an outer diameter thereof. For example, an O-ring or other tool may be used around the folded-over portion(s) of the tube 40 to hold/seal the tube portion to the frame 31. For example, the O-ring can be crushed against the frame 31, thereby sandwiching the tube portion against the frame 31.

The tubular balloon 40 can have a cylindrical toroid shape, with a flow channel 49 running down an axis of the tube 40. Although shown as a cylindrical/elliptical tube/toroid, in some examples, the elastic member/balloon 40 has the shape of a circular torus. The elastic tube 40 can function as an arterial flow optimizer to generate vascular compliance, as described in detail herein. Although described as elastic in some contexts, it should be understood that the tube 40 may be at least partially inelastic. For example, either or both of the inner 41 and outer 44 layers of the tube 40 may be inelastic. In some implementations, the inner layer/tube 41 is elastic to allow for radial stretching/expansion thereof, while the outer layer 44 is inelastic, at least in part.

The chamber(s) 42 of the tubular balloon 40 can be filled with compressible fluid that can compress in the presence of elevated pressure levels (e.g., systolic aortic pressure) and be prone to expand to a greater volume as pressure decreases (e.g., diastolic aortic pressure), wherein such expansion induces blood flow within channel 49 of the tube 40 and thereby in the target blood vessel (e.g., aorta). The fluid 45 can be any suitable or desirable compressible fluid, such as a compressible gas. In some examples, the tubular balloon 40 comprises compressible foam disposed between the inner 41 and outer 44 layers of the balloon 40, and/or between the inner tubular layer 41 and the frame 31. That is, the illustrated medium 45 between the inner 41 and outer 44 layers may comprise foam with compressible gas occupying internal space/pockets thereof. The foam may be configured to provide structure for the balloon 40 in the chamber(s) 42. Compressible gas or other fluid may occupy the pockets of the foam as it expands in lower-pressure conditions; such gas can compress under high-pressure conditions as the foam compresses. For example, the foam may be cylindrical/toroidal in shape and may be coupled (e.g., adhered) to the balloon/tube 40 on an inner and and/or outer diameter of the cylindrical foam structure.

The compliance conduit 49 formed by the compressible/expandable tubular balloon 40 is configured to contract and expand as the heart beat cycles, mimicking the compliance of the vascular tissue. The expansion and/or contraction of the inner tube/layer 41 can be facilitated by the compression of the gas and/or foam 45 disposed within the balloon chamber(s) 42. For example, the tube 40 can be biased towards a certain shape and/or expansion state, such that the expansion of the gas/foam 45 promotes a return to such state/shape after blood pressure causes compression/deformation of the gas/foam 45, thereby introducing/providing compliance to promote blood flow in the blood vessel (e.g., aorta).

The frame 31 may have associated therewith certain to tissue-anchoring features, such as barbs, wires, or the like. For example, the distal end portion(s) of the frame 31 may have wires or barbs having free ends that may be manipulated to be directed radially outward to puncture the tissue of the native blood vessel and secure the frame 31 in place. In some implementations, such wires/barbs may have shape-memory that predisposes such structure to deflect radially outwardly once deployed from a capsule/sheath to facilitate anchoring of the frame 31 to the native blood vessel.

The device 30 described above may be utilized as a compliance-restoration device including at least the outer stent frame 31 and a compressible tubular balloon coupled to/within the frame 31, wherein a fluid conduit 49 runs through the balloon 40. Such devices may have any of the features described or referenced above, including a cylindrical foam structure disposed within the tubular balloon 40, wherein the foam can be attached/adhered to the balloon on an inner and/or outer diameter of the foam, or the foam can be unattached. The balloon 40 can have a circular (e.g., torus) or oval/oblong longitudinal cross-sectional shape (e.g., cylindrical/elliptical toroid, as shown in the relevant figures). The device 30 can be compressible for delivery within a catheter/sheath. The device 30 may be placed in the ascending aorta, such as just about the aortic valve, or in any other position in the aorta or inferior vena cava.

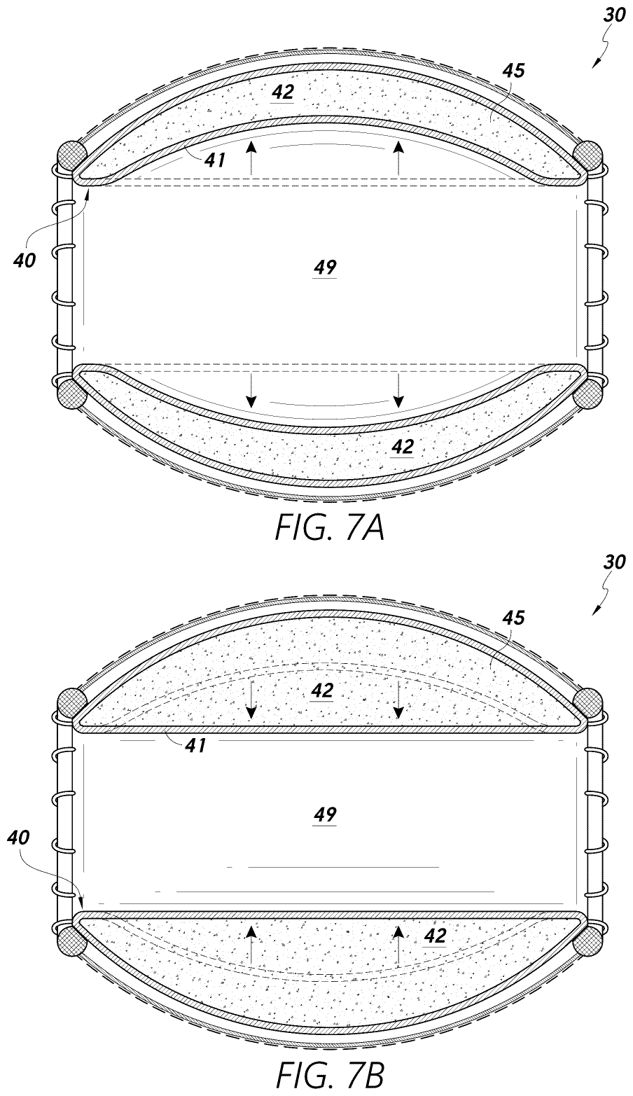

FIGS. 7A and 7B shows side cross-sectional views of the compliance implant device 30 of FIGS. 6A-6E in compressed and relaxed configurations, respectively, in accordance with one or more examples. In the image of FIG. 7A, the flow channel 49 within the tubular balloon 40 is filled with blood/fluid having a higher fluid pressure than that of the medium 45 in the sealed chamber(s) 42 formed around the tubular channel 49. Therefore, the inner tube/layer(s) 41 of the tubular balloon 40 is expanded radially outward, thereby compressing the fluid and/or foam 45 within the chamber(s) 42. As the inner diameter 41 expands outward, the volume of the channel 49 increases and energy is stored in the tubular balloon 40. For example, energy may be stored in the compressed fluid 45 and/or in the inner tube/layer 41, such as in the form of elastic stretch of the material of the inner tube/layer 41. The configuration/state of the implant device 30 in FIG. 7A may be associated with blood pressure conditions associated with the systolic phase of the cardiac cycle.

In the image of FIG. 7B, the stored energy in the tubular balloon 40 causes the inner diameter 41 of the tube 40 to contract/recoil back towards the original cylindrical tubular state thereof due to decreasing fluid pressure within the channel 49. That is, as the pressure gradient between the channel 49 and the chamber(s) 42 decreases, the gas, foam, and/or other medium 45 within the chamber(s) 42 is permitted to expand, thereby causing the inner diameter 41 to collapse, which returns the stored energy in the chamber(s) 42 and/or inner tube/layer(s) 41 to the blood circulation by decreasing the volume of the channel 49. The configuration/state of the implant device 30 in FIG. 7B may be associated with blood pressure conditions associated with the diastolic phase of the cardiac cycle.

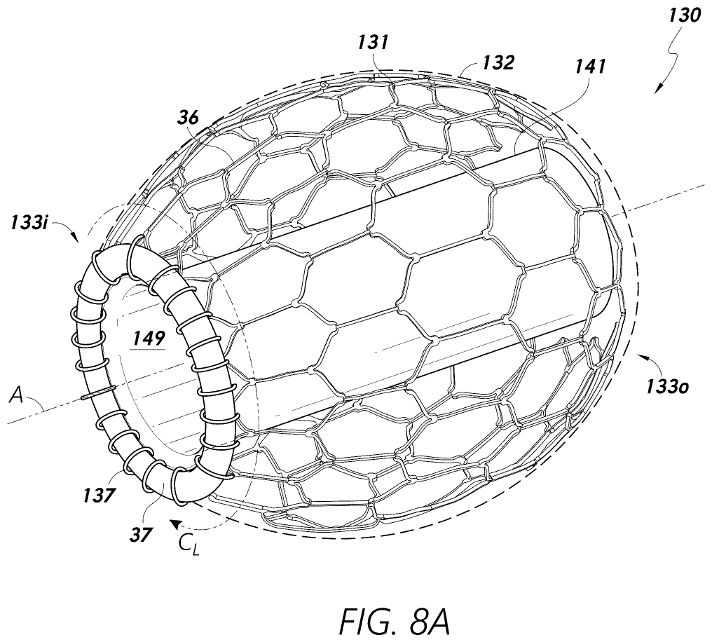

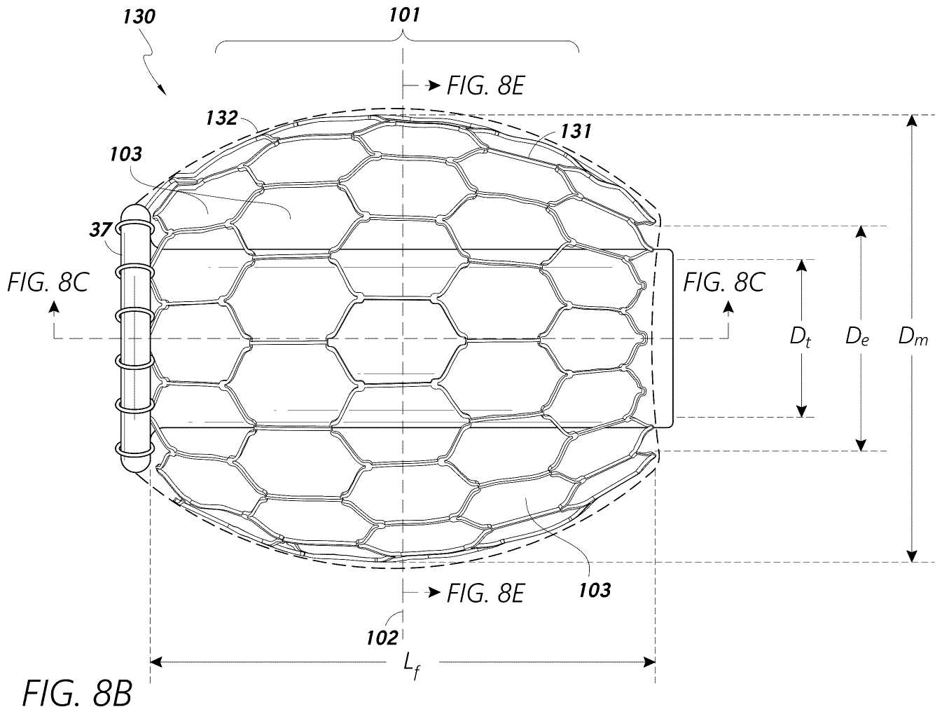

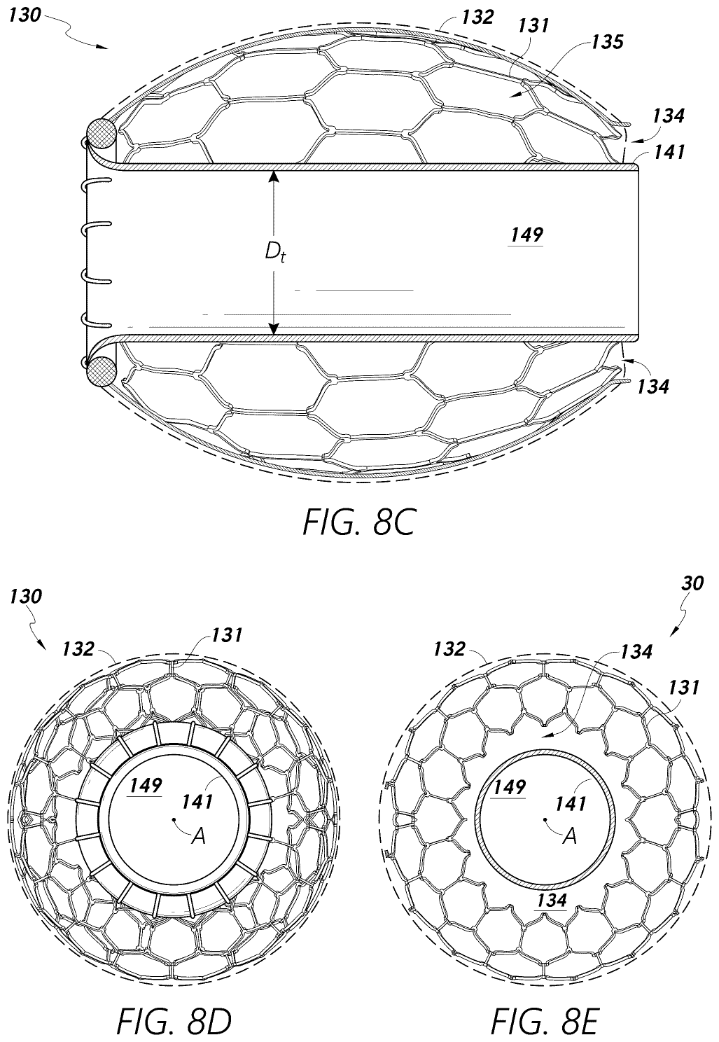

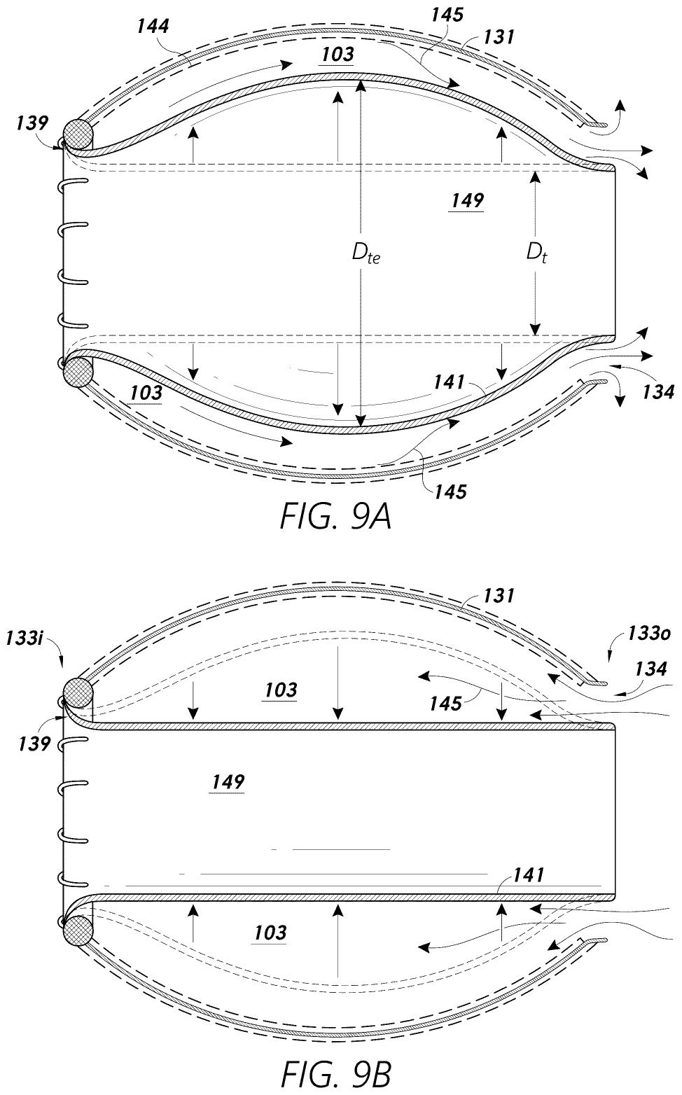

FIGS. 8A-8E provide views of a compliance implant device 130 including an elastic tube 141 in accordance with one or more examples. FIGS. 9A and 9B shows side cross-sectional views of the compliance implant device 130 of FIGS. 8A-8E in compressed and relaxed configurations, respectively. As with other embodiments disclosed herein, the device 130 may form an axial internal central axial fluid channel 149 formed by a tube 141 that is configured to radially expand and retract/recoil based on changing pressure conditions within the channel 149. The radially-expandable tube 141 may have disposed thereabout space defined between the tube 141 and the frame 131, wherein radial expansion of the tube 141, which causes an increase in the volume of the channel 149, causes a commensurate reduction in the volume of the space 103. As the inner tube 141 radially expands and contracts, the volume of the channel 149 increases and decreases in a manner as to provide compliance for the blood vessel in which it is implanted.

The expansive tube 141 can be constructed of a compliant material, such as an elastomeric polymer or other material configured to radially expand/stretch and contract/recoil in response to changing pressure/force conditions. In some examples, the tube 141 comprises a woven structure, such as a woven memory metal braided structure, or the like. In some examples, the tube 141 comprises biological tissue.

The space 103 between the tube 141 (e.g., polymer tube) and the frame 131 may be open on at least one axial end thereof on an outer diameter thereof, such that fluid can flow into the area/space 103 on the outer diameter of the tube 141. That is, the tube 141 may be sealed on one end 139, such that fluid is not permitted to flow between the tube 141 and the frame 131 from such side when the device 130 is implanted in a native blood vessel. Unlike certain other examples disclosed herein, the space 103 may not provide a sealed, gas-filled chamber, but rather may be open in a manner such that fluid/gas can flow into and/or out of the space 103 through one or more openings/accesses 134 on one or both axial ends/sides of the device 130. Therefore, when the device 130 is implanted in the target blood vessel, some amount of blood flow may be inclined to fill the space/103 between the tube 141 and the frame 131. When the tube 141 is in a relaxed, generally-cylindrical state/configuration, blood may enter the space 103 through one or more openings/gaps 134 and collect within the space 130 until it is pushed back out of the device/space 103 in response to radial expansion of the tube 141 in a manner as to encroach into, and reduce the volume of, the space 103. Where the opening/access 134 to the space 103 is on an outflow end/side 1330 of the device 130, the blood collected in the space 103 may be propelled back out of the space 103 generally in the flow direction f of the blood circulation flow.

The compliant channel 149 is defined/formed by the expandable inner tube 141. The inner tube 141 may further serve as a radially-internal diameter/boundary of the blood-filled space 103. The tube 141 may be secured in some manner to the frame 131. For example, the tube 141 may be attached using sutures or other fixation means (e.g., one or more clips, clamps, hooks, loops, adhesives, or the like) at one or both axial ends 33 of the frame and/or tube. In some examples, at one or both axial ends of the tube 141, the tube 141 is stitched circumferentially around the tube 141 to the frame 131 and/or suturing structure/ring coupled to the frame 131.

The frame 131 is configured to expand to an expanded diameter Dₘ (e.g., in the medial area 101 thereof), wherein such expansion of the frame 131 within the native blood vessel (e.g., aorta) may serve to dilate the native blood vessel to some degree. For example, the diameter Dₘ of the frame may advantageously be at least 20% greater than the diameter of the native blood vessel in the area in which the frame is deployed. In some implementations, the diameter Dₘ is more than 30%, 35%, 40%, 45%, or 50% greater than the diameter of the native blood vessel, wherein the frame 131 performs a blood vessel dilation function when implanted/deployed, thereby substantially increasing the diameter of the native blood vessel and maintaining such dilation when the implant 130 is disposed/implanted at the target anatomical site.

The outer frame 131 may have any of the features of the frame 31 discussed above in connection with FIGS. 6A-6E. The outer frames 131 can have a gradually-increasing diameter moving from the ends 133 of the frame towards the axial center 102, as shown, which can provide reduced strain on the native blood vessel tissue compared to examples in which the diameter of the frame is both greater than the native blood vessel and constant along the entire length thereof. Expansion of the frame 131 may be implemented in a gradual and nontraumatic manner. For example, self-expansion or balloon-expansion of the frame 131 may be implemented relatively slowly to allow the native tissue to acclimate and respond gradually to the dilation from its natural diameter to the expanded diameter Dₘ.

The implant device 130 is configured to add back compliance to a target blood vessel in which it is implanted, such as the aorta, to improve perfusion of the heart muscle. The device 130 may be a percutaneously-placeable implant configured to be compressed (e.g., radially compressed) and transported within a delivery catheter or other tubular delivery system. The radially-expandable inner tube 141, in a natural, relaxed, and/or de-pressurized configuration/state can have a straight cylindrical shape/form, whereas in a radially-expanded, pressurized configuration/state, the tube 141 may have an externally-convex (e.g., internally concave) cylindrical shape, which may be similar to the shape of the expanded frame 131 of the device 130. The device 130 may advantageously function as an arterial flow optimizer to generate vascular compliance.

The outer frame 131 may be an expandable stent-type frame configured to expand radially from a compressed delivery configuration to the expanded state shown in FIG. 8A. To achieve this change in the shape and dimension of the frame/stent 131, the frame 131 may have a structure comprising a plurality of struts forming an array of cells 135, which may have any suitable or desirable shape (e.g., oval/ellipse, diamond/rhombus, hexagonal diamond/polygon, etc.). The cells 135 may be arranged in any number of columns in the circumferential direction and rows in the axial, or lengthwise, direction A. In some examples, a short axis of the cells 135 extends in the circumferential direction C and a long axis of the cells 135 extends in the axial direction A, or vice versa. In some examples, adjacent cells 135 are connected to each other in the circumferential and/or axial direction by connecting struts.

The cells 135 of the frame 131 may be formed using any suitable process, such as by stamping or machining the frame structure from a sheet or tube of metal. The frame 131 may be made of any at least partially rigid material, such as metal or plastic. For example, the frame may comprise stainless steel or nitinol. Where nitinol or other shape-memory metal or material is implemented for the frame 131, the frame may be self-expanding. In some implementations, the array of struts is formed from a sheet of metal, which is rolled into a cylinder to form the tubular/cylindrical form of the frame configured for placement within a blood vessel. In some implementations, a balloon catheter can be used to expand the frame 131 for securing in the wall of an artery or other blood vessel or body cavity.

The expandable (e.g., elastic) inner tube/channel 141 is disposed at least partially within the frame 131. The inner member 141 is configured such that at least portion(s) thereof is/are expanded outward towards the frame 131 in response to a pressure gradient between the blood present/flowing through the axial channel 149 of the inner member/tube 141 and the blood 145 collected/disposed within the space 103 between the tube 141 and the frame 131. Such pressure gradient may be due at least in part to the greater flow rate of the blood in the channel 149 compared to the more static blood 145 on the outside of the tube 141. The device 130 may be implanted in, for example, the ascending aorta, such as in an area just above the aortic valve.

The radially-expandable inner tube 141, which is disposed within the cylindrical outer stent frame 131, forms an axial blood flow channel/conduit 149 therethrough. In some examples, the outside of the frame 131 is covered with a fabric or polymer cover 132. The stent frame 131 may be a self-expandable and/or balloon-expandable stent. The frame 131 can have a bulbous outer diameter, such that the diameter thereof is greater in the axial center Dₘ than at the axial ends Dₑ, as shown. Such bulbous/bulging shape can facilitate tighter fit of the device in the target blood vessel in which it is implanted.

The frame 131 may advantageously have a shape that accommodates and facilitates compliant expansion of the inner tube/channel 141. For example, the inner tube 141 may define a flow channel 149 having a diameter Dₜ in a relaxed (e.g., diastolic) configuration/state. For example, the diameter Dₜ of the tube 141 may be approximately equal to the diameter Dₑ of the end portions of the frame 131. As possible example values that can be deviated from while still remaining within the context of the present disclosure, Dₜ may be a value/dimension between approximately 1-2 cm, D_{M} may be a value/dimension between 3-6 cm, and Dₑ may be a value/dimension between 1.5-3 cm in some implementations.

As shown, the frame 131 may have a shape of a convex cylinder, wherein a diameter of the frame 131 is nonuniform along an axis/length L_{f} of the frame 131. For example, as shown, the diameter Dₑ of the frame may be relatively smaller/narrower at one or both ends 133i, 1330 of the frame, wherein the diameter of the frame may increase moving towards an axial/lengthwise center 102 of the frame 131 to the expanded diameter Dₘ. In some examples, the increase in diameter moving towards the axial center 102 may be gradual and/or continuous, as in the illustrated example. However, in some examples, the diameter of the frame may expand in a stepwise manner, such that the maximum diameter Dₘ covers a substantial portion of the length of the frame (e.g., at least half of the length of the frame). For example, the frame 131 may increase in diameter from the narrower frame diameter Dₑ to the expanded frame diameter Dₘ in the end/outer portions/quartiles of the length L_{f} of the frame 131, such that the expanded diameter spans a medial section 101 of the frame, wherein the medial section 101 may span at least 50% of the length L_{f} of the frame 131.

In some applications, the diameter Dₑ of the end portions 133 of the frame 131 may be sized to match the diameter of the native blood vessel in which the frame 131 is deployed. For example, it may be desirable for the diameter Dₑ of the end portions 133 of the frame 131 to be within 20% of the diameter of the native aorta in the particular target segment thereof. The frame 131 is advantageously configured to dilate a target aortic vessel to a diameter at an axial center of the frame that is greater than 20% wider than the respective aortic diameters recited above, while not causing rupturing or undue trauma to the native vessel wall.

The convex cylindrical shape of the frame 131, when combined with the generally straight cylindrical shape/form of the inner tube 141, may advantageously form/provide the space 103 between the frame 131 and the tubular channel 149 defined by the inner tube 141 when the tube 141 is in a relaxed configuration, wherein the tube 141 can expand within the space 103. The inner tube 141 may be configured to cycle between a relaxed state in low-pressure periods (e.g., diastolic phase of the cardiac cycle), wherein the tube 141 is generally cylindrical or slightly convex or concave in the relaxed state, and a radially expanded state in high-pressure periods (e.g., systolic phase of the cardiac cycle), wherein the tube 141 radially expands/bows outward towards the frame 131 within the space 103 provided by the dilation of the blood vessel by the frame 131. That is, the shape and dimensions of the frame 131 advantageously provides a space 103 within which the tube/layer 141 is permitted to radially expand, wherein such expansion may take place within the dilated target blood vessel. By allowing for compliant expansion that is to a diameter that is greater than the diameter of the native blood vessel (e.g., at least 20% greater than the diameter of the native blood vessel prior to expansion thereof by the frame 131), while providing for such expansion within the native blood vessel without requiring grafting, cutting, and/or resection of the native blood vessel (e.g., aorta), risks associated with blood vessel rupture can be reduced or eliminated. Furthermore, by dilating the blood vessel to allow for the expanding tube 141 to be contained within the blood vessel, in the event that the tube 141 leaks or ruptures in some manner, such leakage may be maintained substantially within the target blood vessel. Such leakage within the venous blood vessel may result in relatively less damage/injury to the patient compared to blood flow leakage outside of the circulatory system within the body cavity. For example, rupture of the tube 141 within the target blood vessel may result in substantially no damage or injury to the patient.

In some examples, the space 103 is primarily occupied by blood 145 between the inner tube 141 and the frame 131 (and/or polymer or other layer 144 lining the inner diameter of the frame 131), wherein radial expansion of the inner tube 141 causes expulsion of at least a portion of the blood 145 from one or more openings or channels 134 on the outside of the tube 141 at the outflow (and/or inflow) end of the device 130.

Without dilation of the native blood vessel, as effected by the frame 131, the space available within the frame for blood flow and expansion of the inner tube/layer 141 would be limited. For example, if the frame 131 had a constant diameter that was only slightly larger than the diameter Dₜ of the tubular channel 149, the inner tube 141 would be unable to radially expand by a significant or effective amount. Therefore, the ability of the tube 141 to change in volume cyclically in a manner as to add compliance to the blood circulation would be limited. That is, the convex cylindrical shape of the frame 131, wherein the frame has, at its maximum diameter Dₘ, a diameter that is at least 20%, 30%, 40%, and/or 50% greater than the diameter of the native blood vessel, increases the ability of the inner tube 141 to add compliance to the blood circulation.

The outer frame 131 may be at least partially covered with a fabric or polymer coating or cover 132, which may promote tissue ingrowth with the inner diameter of the native blood vessel and/or provide a fluid-sealing function. The cover 132 may be disposed on an outer surface or area of the frame 131, and/or may be disposed/applied to the inner diameter of the frame 131 on an inside thereof. For example, the layer(s) of covering 131 may be disposed in some implementations between the outer frame 131 and the tube 141. In some implementations, the cover 132 comprises a cloth or polymer sleeve which may be at least partially elastic, or alternatively nonelastic. The covering 132 may be applied over and/or within the frame 131 in any suitable or desirable manner. For example, in some implementations, the cover 132 may be applied using an electrical or mechanical spinning (e.g., rotary jet spinning, electrospinning, or similar) application process or other deposition process known to those having ordinary skill in the art.

The frame 131 may be a self-expandable and/or balloon-expandable stent frame. As described above, the frame 131 may have a bulbous outer diameter, such that the diameter thereof is substantially greater in the axial center 102 than at one or more of the axial ends 133 thereof, as shown. Such bulbous/bulging shape can facilitate tighter fit of the device in the blood vessel in which it is implanted. The frame 131 can include barbs or other anchors, as shown in the example of FIG. 6A and described above, associated with an outer diameter thereof to engage the target native blood vessel wall and secure the device in place. Such barbs/anchors may puncture through an outer cover/coating 132 and into the native tissue to anchor the frame 131 in place. In some examples, the distal end portion(s) 133 of the frame 131 may have wires or barbs having free ends that may be manipulated to be directed radially outward to puncture the tissue of the native blood vessel and secure the frame 131 in place. In some implementations, such wires/barbs may have shape-memory that predisposes such structure to deflect radially outwardly once deployed from a capsule/sheath to facilitate anchoring of the frame 131 to the native blood vessel.

The compliance conduit 149 formed by the elastic tube 141 is configured to contract and expand as the heart beat cycles, mimicking the compliance of the vascular tissue. The expansion and/or contraction of the inner tube 141 can be facilitated by the changing pressure gradient between the blood in the channel 149 and the blood in the space 103 outside the tube 141. For example, the tube 141 can be biased towards the relaxed cylindrical shape shown in FIG. 8A, such that a decrease in the pressure gradient promotes a return to such state/shape after blood pressure inside the channel 149 causes expansion of the tube 141, thereby introducing/providing compliance to promote blood flow in the blood vessel (e.g., aorta).

The implant device 130 may or may not include a layer 144 of material on the inner diameter of the frame 131. Where such layer is present, axial sealing of the space 103 may be implemented through integration or sealing of the tube 141 with/to the outer layer 144. Such layer 144 may comprise the same material as the tube 141 and may be an integrated form therewith. For example, the tube 141, together with the outer layer 144 may provide a two-layer implementation with a chamber 103 formed therebetween, which may be similar in some respects to the gas-chamber examples illustrated and described above, except that the chamber(s) 103 may have fluid access opening(s)/channel(s) 134, rather than being fluidly sealed as in the embodiments of FIGS. 6 and 7. The implementation of a layer 144 on the inner diameter of the frame 131 may provide protection against contact with the frame by the inner tube 141, which may cause tearing or other abrasion or damage thereto. The layer(s) 144 may provide a liner for protection or other purposes. For example, the layer 144 and provide fluid-sealing characteristics, which may be desirable in some embodiments to prevent pass-through of fluid from the space 103 out of the device 130 through the cells of the frame 131.

In the image of FIG. 9A, the flow channel 149 within the elastic tube 141 is filled with blood/fluid having a higher fluid pressure than that of the blood/fluid 145 disposed radially outside of the tube 141 in the space 103. Therefore, as shown, the inner tube 141 is expanded radially outward, thereby reducing the volume of the space 103 between the outer diameter of the tube 141 and the frame 131 and pushing/expelling the blood/fluid 145 contained therein out of the space 103 and into the circulation downstream of the implant device 130. As the inner diameter 141 expands outward to the expanded diameter Dₜₑ, the volume of the channel 149 increases and energy is stored in the tube 141. For example, energy may be stored in the form of elastic stretch of the material of the inner tube 141. The configuration/state of the implant device 130 in FIG. 9A may be associated with blood pressure conditions associated with the systolic phase of the cardiac cycle.

In the image of FIG. 9B, the stored energy in the tube 141 causes the tube 141 to contract/recoil back towards the original cylindrical tubular state thereof due to decreasing blood/fluid pressure within the channel 149 relative to the pressure in the blood/fluid 145 radially outside of the tube 141 in the space 103. That is, as the pressure gradient between the channel 149 and the space 103 decreases, the tube 141 collapses, which returns the stored energy in the tube 141 to the blood circulation. As the tube 141 retracts/returns to the cylindrical shape, the volume of the channel 149 decreases, which causes the increase in flow through the channel 149. In addition, as the tube contracts, the volume of the space 103 between the tube 141 and the frame 131 increases, which may cause a vacuum effect that draws blood from within the blood vessel into the space 103, as shown. Due to the different fluid dynamics of the blood 145 that is sucked into the space 103 compared to the blood circulation within the channel 149, the pressure in the blood 145 in the space 103 may be lower than the pressure of the blood within the channel 149 during at least portion(s) of the cardiac cycle, wherein such gradient may advantageously enable the expansion and contraction of the tube 141 to provide the increase in compliance described in detail herein. The configuration/state of the implant device 130 in FIG. 9B may be associated with blood pressure conditions associated with the diastolic phase of the cardiac cycle.

By including openings/access 134 into the space 103 outside of the tube 141, the pressure gradient between the space 103 and the channel 149 may be less than certain other examples in which fully-sealed gas chambers are disposed about the flow channel due to the fluid communication between the blood in the channel 149 and the space 103. Therefore, implementation of sealed gas chambers and compliance restoration devices in accordance with aspects of the present disclosure may be preferable in some instances and/or with respect to certain patients. However, the simplicity of the design of the device 130 shown in FIGS. 8 and 9 can provide certain benefits that may outweigh other considerations. Furthermore, as the device 130 does not include sealed gas chambers, risks associated with rupture and/or outgassing from such chamber(s) may be avoided. The opening(s) 134 into the space 103 around the tube 141 may have any suitable or desirable form more features. For example, some number of sutures or connections between the tube 141 of the frame 131 may be present at the end through which the opening(s)/passage(s) 134 provide access, wherein gaps between such sutures/connections provide the access opening(s).

The device 130 is shown and described as having opening(s) into the space 103 on the outflow end/side 1330 thereof. However, should be understood that in some implementations, one or more openings may be present on the inflow side 133i. In such examples, the outflow end 1330 may be sealed-off to prevent fluid entry from the outflow end/side, or one or more openings may be present into and out of the space 103 from the inflow 133i and outflow 1330 sides, wherein some amount of blood may flow into the space 103 through the inflow and/or outflow ends and/or may be expelled from the space 103 through one or both ends. In the examples in which the space 103 is sealed on the outflow end 1330 but at least partially open on the inflow and 133i, expulsion from the space 103 of blood/fluid due to expansion of the tube 141 and channel 149 may result in flow of fluid out of the space 103 that moves against the natural flow f of the circulation. In some cases, such upstream flow may be desirable as a mechanism for further controlling/altering the flow of blood in the target blood vessel and/or upstream of the implant 130. In such examples, as the tube 141 contracts/recoils, blood may be drawn into the space 103 from the inflow end 133i, which may further alter flow characteristics of the circulation through the blood vessel.

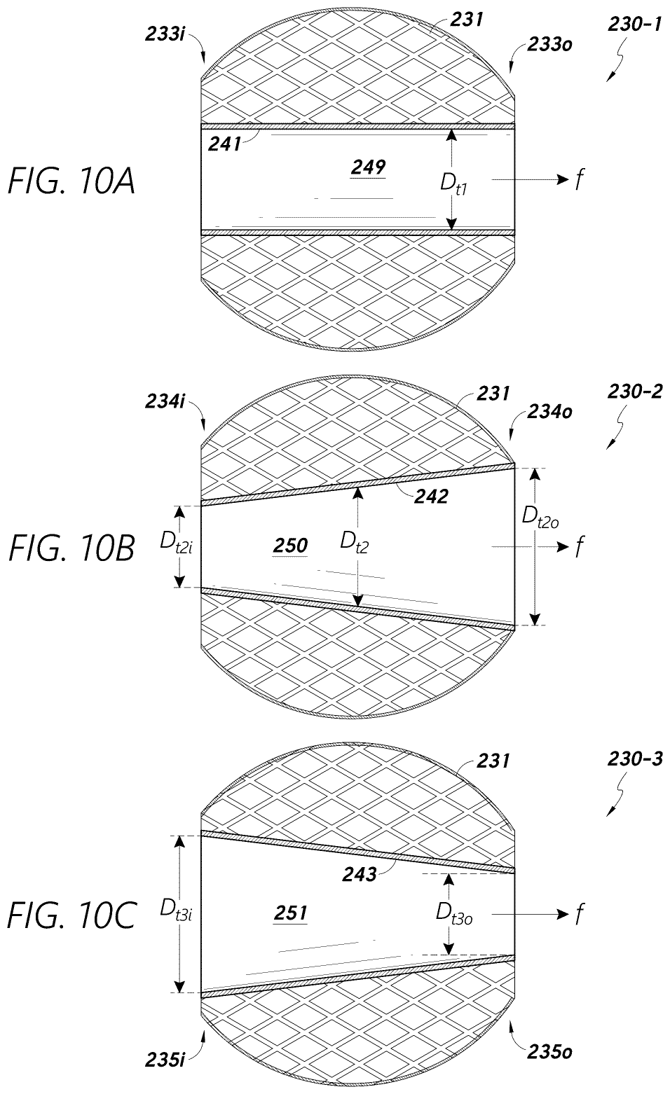

FIGS. 10A, 10B, and 10C show cross-sectional side views of compliance implant devices 230-1, 230-2, 230-3 having respective flow channel configurations in accordance with various examples. The flow channels of the devices shown in FIGS. 10A, 10B, and 10C may be formed by the configurations of the respective expandable/elastic tubes 241, 242, 243 thereof, which may have any of the features of the various compliance-enhancing tubes disclosed herein,

FIG. 10A shows a compliance implant device 230-1 including an elastic tube 241 having a generally-cylindrical/straight shape, where in the diameter Da of the tube 241 and channel 249 formed thereby is continuous/uniform along a length Z of the device 230-1 and/or tube 241, at least when the tube 241 is in a relaxed/non-expanded state or configuration as shown in FIG. 10A.

In FIG. 10B, a compliance-enhancement implant device 230-2 is shown that comprises an expandable inner tube 242 that has a diameter Dₜ₂ that is nonuniform along the length L of the device, at least when the tube 242 is in a relaxed/non-expanded state or configuration as shown. For example, the diameter Dₑ of the tube 242 and flow channel 250 may expand in a continuous or non-continuous manner moving from an inflow end 234i to an outflow end 2340 of the device 230-2. For example, as shown, the diameter Dₜ₂ᵢ in an area of the inflow end 234i may be less than the diameter Dₜ₂ₒ associated with the outflow end/portion 2340. In order to achieve the expanding diameter of the tube 242, the tube 242 may comprise a frustoconical shape that flares outward towards the outflow end 234o and/or in the direction of flow f through the channel 250, as shown. The flared/expanding shape/form of the tube 242 may affect fluid dynamics of circulation flow through the channel 250. For example, the increase in diameter/volume of the tube 242 and channel 250 towards the outflow end 234o may result in reduced flow velocity and/or pressure at or near the outflow end 234o relative to the inflow end 234i of the channel 250, which may be desirable as a mechanism for controlling flow of the blood circulation.

In FIG. 10C, a compliance-enhancement implant device 230-3 is shown that comprises an expandable inner tube 243 that has a diameter Dₜ₃ that is nonuniform along the length L of the device, at least when the tube 243 is in a relaxed/non-expanded state or configuration as shown. For example, the diameter Dₜ₃ of the tube 243 and flow channel 251 may decrease in a continuous or non-continuous manner moving from an inflow end 235i to an outflow end 2350 of the device 230-3. For example, as shown, the diameter Dₜ₃ᵢ in an area of the inflow end 235i may be greater than the diameter Dₜ₃ₒ associated with the outflow end/portion 2350. In order to achieve the decreasing diameter of the tube 243, the tube 243 may comprise a frustoconical shape that flares outward towards the inflow end 235i and/or against the direction of flow f through the channel 251, as shown. The flared/reducing shape/form of the tube 243 may affect fluid dynamics of circulation flow through the channel 251. For example, the decrease in diameter/volume of the tube 243 and channel 251 towards the outflow end 2350 may result in increased flow velocity and/or pressure at or near the outflow end 2350 relative to the inflow end 235i of the channel 251, which may be desirable as a mechanism for controlling flow of the blood circulation.

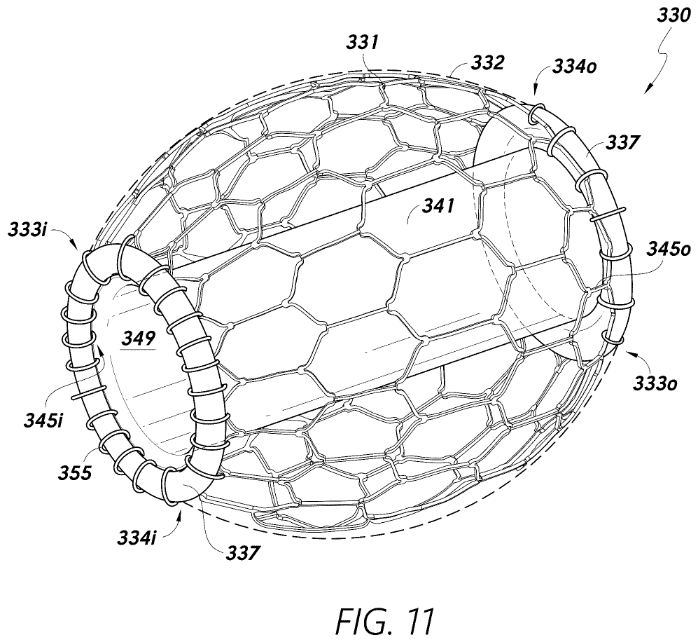

FIG. 11 shows a compliance implant device 330 including an internal elastic tube 341 that is sutured at inflow 333i and/or outflow 3330 ends thereof to an outer frame 331 in accordance with one or more examples. That is, one or more sutures, or loops of suture, 355 may be utilized to suture the tube 341 to the frame 331, such as directly to end portion(s) 333 of the frame 331 or to a suture ring or other structure coupled to the end portion(s) 333 of the frame 331. The tube 341 forms a central flow channel 349.

The compliance-enhancement tube 341 can be coupled to the frame 331 at one or both axial ends 333 of the frame 331. For example, the tube 341 can be sutured to respective ring structures 337 at either or both axial ends 333 of the stent frame 331. Where/when implemented, the ring structure(s) 337 may be integrated with the frame 331 or may be attached to one or both axial ends 333 thereof in some manner. For example, sutures, wires, or other coupling means 355 may be used to attach the ring(s) 337 to the frame 331.

The expandable tube 341 may further be sutured or otherwise secured/attached to the axial end(s) 333 of the frame 331. For example, the tube 341 may be sutured to the suture ring 337 or other structure of the frame 331 (e.g., to strut(s) of the frame 331), or may be attached using an adhesive or other attachment means. In some implementations, end portion(s) 345 of the tube 341 may be heat-adhered or otherwise sealed to the frame 331 and/or ring(s) 337 a manner such that a fluid barrier is present at the interface of the tube 341 with the frame 331. In some implementations, gaps may be present between adjacent suture loops 355 that allow for fluid to flow into and/or out of the space between the tube 341 and the frame 331 at axial ends thereof. The device 330 may have the tube 341 and frame 331 sutured/coupled at just the inflow end 334i of the device 330, at just the outflow end 3340, at both the inflow 334i and outflow 3340 ends, or at neither end. The device 330 may have a cover 332 outside and/or inside of the frame 331.

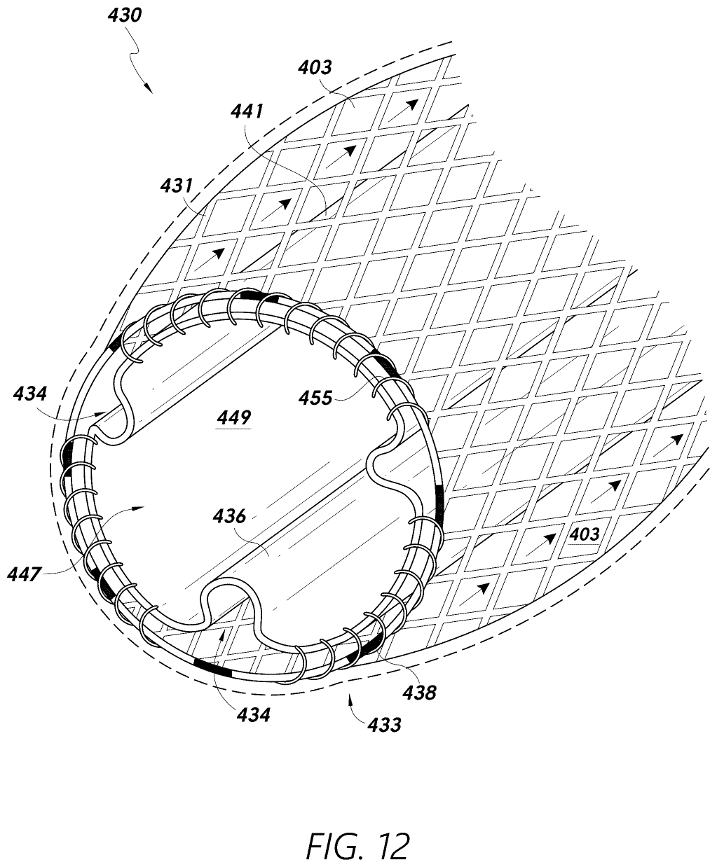

FIG. 12 shows a compliance implant device 430 including an internal elastic tube 441 that has one or more circumferential inlets/outlets 434 in accordance with one or more examples. The tube of material 441 may have a perimeter/circumference thereof that is greater than the circumference/perimeter of the inlet or outlet 447, such that the material of the tube can be inwardly projected to form the inlet/outlet channel(s) 434. The inlet(s) 434 may be associated with a lengthwise channel 436 that runs at least a portion of the length of the tube 441. For example, the inlets 434 may provide access to the lengthwise channel(s) 436, which may allow for the passage of blood through into and/or through space 403 between the tube 441 and the outer frame 431. In some implementations, blood flow into and/or through the channel(s) 436 comprises most or all of the blood flowing or present within the space 403 outside of the tube 441. The tube 441 forms a central flow channel 449.

The inlet/outlet features 434 may be formed by suturing the tube 441 around the perimeter thereof to the frame 431 and/or other feature of the device 430 at inflow and/or outflow ends, wherein gaps in the suturing 455 are present in the areas of the inlets/outlets 434 to provide gaps for flow into and/or out of such features 434. With respect to the device 430, it should be understood that the features illustrated may be associated with either or both of inlet and outlet ends 433 of the device 430. Where features 434 are associated with both inlet and outlet ends 433 of the device 430, blood may be permitted to flow through the space 403 entering on an inlet end of the device 430 and exiting at an outlet thereof. The features 434 may allow for inlet and/or outlet flow of blood in/through the space 403 in a manner as to reduce the risk of blood stagnation and/or occurrence/formation of blood clots/embolus within the space 403. Although the illustration of FIG. 12 shows blood flow through a defined channel 436 that traverses a length of the device 430, in some implementations, no such channels exist, but rather blood flowing into the space 43 through the feature(s) 434 may enter a circumferentially-open chamber/space 403 that is not defined by longitudinal channel(s). The device 430 may comprise one or more radiopaque markers 438 to assist with visualization of the device during and/or after implantation.

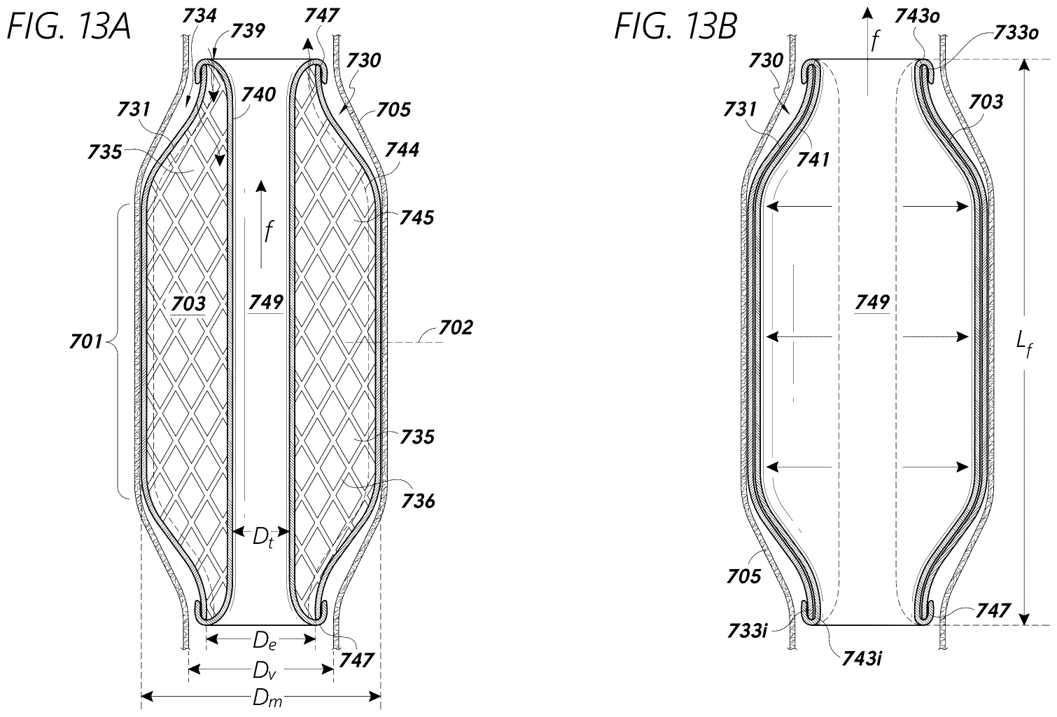

FIGS. 13A and 13B show views of a compliance implant device 730 including a tubular structure 741, such as an elastic sleeve or balloon component, having an axial internal/central fluid channel 749 formed by the tube 741, wherein the tube 741 is configured to radially expand and retract/compress based on changing pressure conditions within the channel 749 and/or in a space between the tube 741 and an outer frame 731 to which the tube 741 is coupled. Blood is received in the channel 749, wherein such blood may have a pressure that is greater than the blood in the space 703, causing the tube 741 to expand radially outwardly, thereby pushing blood out of the space 703.

The elastic sleeve 741 can be attached to the frame 731 at either or both of the upstream 733i and downstream 7330 ends of the frame. The implant device 730 can be deployed within the aorta 705, such as within the descending aorta, or within any other blood vessel segment. The mid/medial portion 701 of the stent/frame 731 is configured to expand away from the sleeve/tube 741, thereby pushing the blood vessel (e.g., aorta) wall radially outward, thereby creating a space 703 between the blood vessel wall 705 (and the stent/frame 731) and the sleeve/tube 741. The attachment of the sleeve/tube 741 to the stent/frame 731 at the ends of the implant 730 can seal the sleeve/tube 741 to the blood vessel, such as by pressing the sleeve/tube 741 against the blood vessel wall 705.

During, for example, ventricular systole, the sleeve/tube 741 can expand outward toward the stent/frame 731 and blood vessel wall 705, wherein the sleeve/tube 741 can elastically contract radially inwardly during, for example, ventricular diastole, thereby simulating compliant, healthy vasculature. Delivery of the implant device 730 to the target anatomy can be made, for example, through transfemoral or other transcatheter access.

The space 703 defined between the tube 741 and the frame 731 can provide an area for radial expansion of the tube 741, which causes an increase in the volume of the channel 749. As the volume of the channel 749 increases, the volume of the space 703 is reduced, as shown in the comparison between FIGS. 13A and 13B. The radial expansion and contraction of the tube 741 causes the volume of the channel 749 to increase and decrease in a manner as to provide compliance characteristics to the blood vessel 705 in which it is implanted.

The expansive tube 741 can be constructed of a compliant material, such as an elastomeric polymer or other material configured to radially expand and contract/recoil in response to changing pressure/force conditions. In some examples, the tube 741 comprises a woven structure, such as a woven memory metal braided structure, or the like. In some examples, the tube 741 comprises biological tissue.

The space 703 between the tube 741 (e.g., polymer tube) and the frame 731 may be open on at least one axial end thereof such that fluid can flow into and out of the area/space 703 on the outer diameter of the tube 741. Such opening(s) 739 may comprise holes or other apertures in the tube 741 in the area of the upstream/inflow or downstream/outflow axial end portions 743 of the tube 741. That is, the tube 741 may be coupled on one or both axial ends 733 to the respective end(s) 733 of the frame 731, wherein fluid flow into and/or out of the space 703 is prevented or inhibited except through the apertures 739. In some examples, no apertures in the tube 741 are included, and rather compressible gas or fluid is maintained in the space 703 in a manner as to store energy when compressed due to expansion of the tube 741 and return energy to the circulation as the pressure in the channel 749 subsides and the medium disposed in the space 703 expands.

In some implementations, when the device 730 is implanted in the target blood vessel 705, some amount of blood flow may be inclined to fill the space 703 between the tube 741 and the frame 731. Such fluid passage into and out of the space 703 may be through apertures or other openings 739 in the tube 741. Additionally or alternatively, fluid ingress/egress may be through open cells of the frame 731 on an outside of the device 730, such as in an area that is radially outside of the tube 741 and/or outside of the coupling of the tube 741 to the frame 731 at distal end(s) 743 thereof, and between the inner diameter of the blood vessel wall 705 and the outer diameter of the frame 731. For example, at least one of the ends 733 of the frame 731 may have a diameter that is less than the diameter of the native blood vessel 705, such that a space between the end of the frame 733 and the native blood vessel is formed at one or more axial ends of the frame 731. When the tube 741 transitions to a relaxed, generally-cylindrical state/configuration, as shown in FIG. 13A, blood may enter the space 703 through one or more openings/gaps 739, 734 and collect within the space 703 until it is pushed back out of the device/space 703 in response to radial expansion of the tube 741 in a manner as to encroach into and reduce the volume of the space 703, as shown in FIG. 13B. Where the opening/access 739 to the space 703 is on an outflow end/side of the device 730, the blood collected in the space 703 may be propelled back out of the space 703 generally in the flow direction f of the blood circulation flow.

The frame 731 may comprise a relatively long stent, which may have a length L_{f} of approximately 10-15 cm, or longer. The tube/balloon 741 may be relatively narrow; the relaxed diameter Dₜ, for example, of the tube 741 in the medial area 701 may be less than the diameter Dₑ of the end(s) of the frame 731 and/or the diameter Dᵥ of the native blood vessel 705 in the area immediately outside of the device 730 with respect to the lengthwise dimension of the device. For example, the diameter Dₜ may be at least 20% less than the diameter Dₑ and/or the diameter Dᵥ of the blood vessel. The diameter Dₜ of the tube may be between 10-15 mm, such as between 8-12 mm. In some implementations, the diameter Dₜ is about 10 mm. The stent diameter Dₘ at least in the medial area 701 may be substantially greater than the diameter Dₜ of the tube 741 in the expanded configuration of the frame 731 shown, such as between 15-25 mm. For example, the expanded diameter Dₘ of the frame 731 may be between 18-22 mm, such as about 20 mm. High pressure conditions can increase the diameter of the tube 741 at least in the medial area 701 to a dimension between Dₜ and Dₘ, such as up to the diameter Dₘ (e.g., 20 mm). The expanded diameter Dₘ of the frame 731 can serve both to secure the device 730 in place within the blood vessel and to provide the expansion space 703.

The compliant channel 749 is defined/formed by the expandable inner tube 741. The inner tube 741 may further serve as a radially-internal diameter/boundary of the space 703. The tube 741 may be secured in some manner to the frame 731. For example, the tube 741 may be attached using sutures or other fixation means (e.g., one or more clips, clamps, hooks, loops, rings, adhesives, thermal adhesion, or the like) at one or both axial ends 733/743 of the frame and/or tube. In some examples, on one or both axial ends of the tube 741, the tube 741 is stitched circumferentially around the tube 741 to the frame 731 and/or suturing structure/ring coupled to the frame 731 and/or tube 741. The tube 741 may advantageously have a length that is greater than an expanded length of the frame 731, such that ends/lips/cuffs 747 of the tube 741 can curl over/around the end(s) 733 of the frame, as shown, and secured to the frame 731 in such configuration. For example, an O-ring or similar device may be placed and secured over the lip/cuff 747 of the tube 741 around the end 733 of the frame 731 and tightened and/or crimped in such position so as to secure the tube 741 to the frame 731. Such coupling of the tube 741 to the frame 731 using cuffs 747 of the tube 741 can improve coupling and/or sealing between the frame 731 and the tube 741.

The frame 731 is configured to expand to the expanded diameter Dₘ (e.g., in the medial area 701 thereof), wherein such expansion of the frame 731 within the native blood vessel 705 (e.g., aorta) may serve to dilate the native blood vessel to some degree. For example, the diameter Dₘ of the frame may advantageously be at least 20% greater than the diameter of the native blood vessel 705 in the area in which the frame 731 is deployed. In some implementations, the diameter Dₘ is more than 25%, 30%, 35%, 40%, 45%, or 50% greater than the diameter Dᵥ of the native blood vessel 705, wherein the frame 731 performs a blood vessel dilation function when implanted/deployed, thereby substantially increasing the diameter of the native blood vessel and maintaining such dilation when the implant 730 is disposed/implanted at the target anatomical site.

The outer frames 731 can have a gradually-increasing diameter moving from the ends 733 of the frame towards the axial center 702, as shown, which can provide reduced strain on the native blood vessel tissue 705 compared to examples in which the diameter of the frame is both greater than the native blood vessel and constant along the entire length L_{f} thereof. In some examples, as in the illustrated implementation, the frame 731, in the expanded configuration shown in FIG. 13A, may have a stepped-out diameter, wherein the diameter of the frame transitions from a narrower diameter Dₑ at the ends 733 to the expanded diameter Dₘ in the medial segment 701 relatively close to the ends 733, such as within the first 20-30% of the length of the frame 731 (e.g., the end quartile of the length) from either end moving to the center 702. The narrowing of the diameter Dₑ at the ends of the frame 731 can advantageously provide a relatively narrower portion of the frame for fixation of the axial ends 743 of the tube 741 thereto, and also may provide a more gradual expansion/dilation of the blood vessel, which may be less traumatic to the blood vessel.

Expansion of the frame 731 may be implemented in a gradual and nontraumatic manner. For example, self-expansion or balloon-expansion of the frame 731 may be implemented relatively slowly to allow the native tissue to acclimate and respond gradually to the dilation from its natural diameter Dᵥ to the expanded diameter Dₘ.

The device 730 may be a percutaneously-placeable implant configured to be compressed (e.g., radially compressed) and transported within a delivery catheter or other tubular delivery system. The radially-expandable inner tube 741, in a natural, relaxed, and/or de-pressurized configuration/state can have a straight cylindrical shape/form, whereas in a radially-expanded, pressurized configuration/state, the tube 741 may have an externally-convex (e.g., internally concave), axially-bulging cylindrical shape, which may be similar to the shape of the expanded frame 731 of the device 730 and may conform thereto as the tube 741 expands. The device 730 may advantageously function as an arterial flow optimizer to generate vascular compliance.

The outer frame 731 may be an expandable stent-type frame configured to expand radially from a compressed delivery configuration to the expanded state shown in FIGS. 13A and 13B. To achieve this change in the shape and dimension of the frame/stent 731, the frame 731 may have a structure comprising a plurality of struts forming an array of cells 735, which may have any suitable or desirable shape (e.g., oval/ellipse, diamond/rhombus, hexagonal diamond/polygon, etc.). The cells 735 may be arranged in any number of columns in the circumferential direction and rows in the axial, or lengthwise, direction A. In some examples, a short axis of the cells 735 extends in the circumferential direction and a long axis of the cells 735 extends in the axial direction A, or vice versa. In some examples, adjacent cells 735 are connected to each other in the circumferential and/or axial direction by connecting struts 736.

The cells 735 of the frame 731 may be formed using any suitable process, such as by stamping or machining the frame structure from a sheet or tube of metal. The frame 731 may be made of any at least partially rigid material, such as metal or plastic. For example, the frame may comprise stainless steel or nitinol. Where nitinol or other shape-memory metal or material is implemented for the frame 735, the frame may be self-expanding. In some implementations, the array of struts is formed from a sheet of metal, which is rolled into a cylinder to form the tubular/cylindrical form of the frame configured for placement within a blood vessel. In some implementations, a balloon catheter can be used to expand the frame 731 for securing in the wall of an artery or other blood vessel or body cavity.

The expandable (e.g., elastic) inner tube/channel 741 is disposed at least partially within the frame 731. The inner member 741 is configured such that at least portion(s) thereof expand outward towards the frame 731 in response to a pressure gradient between the blood present/flowing through the axial channel 749 of the inner member/tube 741 and the blood or other media 745 collected/disposed within the space 703 between the tube 741 and the frame 731. Such pressure gradient may be due at least in part to the greater flow rate of the blood in the channel 749 compared to the more static blood/medium 745 on the outside of the tube 741. The device 730 may be implanted in, for example, the ascending aorta, such as in an area just above the aortic valve, or in the thoracic or abdominal aorta.

The radially-expandable inner tube 741, which is disposed within the cylindrical outer stent frame 731, forms an axial blood flow channel/conduit 749 therethrough. In some examples, the outside of the frame 735 is covered with a fabric or polymer cover. The stent frame 735 may be a self-expandable and/or balloon-expandable stent. The frame 735 can have an axially bulging/bulbous outer diameter, such that the diameter thereof is greater in the axial center Dₘ than at the axial ends Dₑ, as shown. Such bulbous/bulging shape can facilitate tighter fit of the device in the target blood vessel 705 in which it is implanted.

The frame 731 may advantageously have a shape that accommodates and facilitates compliant expansion of the inner tube/channel 741. For example, the inner tube 741 may define a flow channel 749 having a diameter Dₜ in a relaxed (e.g., diastolic) configuration/state. For example, the diameter Dₜ of the tube 741 may be approximately equal to the diameter Dₑ of the end portions 733 of the frame 731, or the diameter Dₜ of the tube 741 may be approximately equal to the diameter Dₑ of the end portions 733 of the frame 731. As possible example values that can be deviated from while still remaining within the context of the present disclosure, Dₜ may be a value/dimension between approximately 0.5-2 cm, Dₘ may be a value/dimension between 3-6 cm, and Dₑ may be a value/dimension between 1.5-3 cm in some implementations.

As shown, the frame 731 may have a shape of a convex cylinder, wherein a diameter of the frame 731 is nonuniform along one or more portions of an axis/length L_{f} of the frame 731 and/or uniform along one or more portions of the length L_{f}, such as over at least a portion of the medial section 701. For example, as shown, the diameter Dₑ of the frame may be relatively smaller/narrower at one or both ends 733i, 7330 of the frame, wherein the diameter of the frame may increase moving towards an axial/lengthwise center 702 of the frame 731 to the expanded diameter Dₘ. In some examples, the diameter of the frame 731 may expand in a stepwise manner, such that the maximum diameter Dₘ covers a substantial portion of the length L_{f} of the frame (e.g., at least half of the length of the frame), as in the illustrated example. For example, the frame 731 may increase in diameter from the narrower frame diameter Dₑ to the expanded frame diameter Dₘ in the end portions/quartiles of the length L_{f} of the frame 731, such that the expanded diameter spans a medial section 701 of the frame. However, in some examples, the increase in diameter moving towards the axial center 702 may be gradual and/or continuous.

In some applications, the diameter Dₑ of the end portions 733 of the frame 731 may be sized to match the diameter Dᵥ of the native blood vessel in which the frame 731 is deployed. For example, it may be desirable for the diameter Dₑ of the end portions 733 of the frame 731 to be within 20% of the diameter Dᵥ of the native aorta 705 in the particular target segment thereof. The frame 731 is advantageously configured to dilate the target vessel (e.g., aorta) 705 to a diameter at an axial center 702 of the frame 731 that is greater than 20% wider than the respective aortic diameters recited above, depending on the particular aortic segment in which the device 730 is implanted, while not causing rupturing or undue trauma to the native vessel wall.

The bulging/convex cylindrical shape of the frame 731, when combined with the generally straight cylindrical shape/form of the inner tube 741, may advantageously provide the space 703 between the frame 731 and the tubular channel 749 defined by the inner tube 741 when the tube 741 is in a relaxed configuration, wherein the tube 741 can radially expand within the space 703. The inner tube 741 may be configured to cycle between a relaxed state in low-pressure periods (e.g., diastolic phase of the cardiac cycle), wherein the tube 741 is generally cylindrical or slightly convex or concave in the relaxed state, and a radially expanded state in high-pressure periods (e.g., systolic phase of the cardiac cycle), wherein the tube 741 radially expands/bows outward towards the frame 731 within the space 703 provided by the dilation of the blood vessel by the frame 731. That is, the shape and dimensions of the frame 731 advantageously provide a space 703 within which the tube/layer 741 is permitted to radially expand, wherein such expansion may take place within the dilated target blood vessel. By allowing for compliant expansion that is to a diameter that is greater than the diameter of the native blood vessel (e.g., at least 20% greater than the diameter of the native blood vessel prior to expansion thereof by the frame 731), while providing for such expansion within the native blood vessel without requiring grafting, cutting, and/or resection of the native blood vessel (e.g., aorta), risks associated with blood vessel rupture can be reduced or eliminated. Furthermore, by dilating the blood vessel to allow for the expanding tube 741 to be contained within the blood vessel, in the event that the tube 741 leaks or ruptures in some manner, such leakage may be maintained substantially within the target blood vessel. Such rupture within the venous blood vessel may result in relatively less damage/injury to the patient compared to rupture causing blood flow leakage outside of the circulatory system within the body cavity. For example, rupture within the target blood vessel 705 may result in substantially no damage or injury to the patient.

In some examples, the space 703 is primarily occupied by blood 745 between the inner tube 741 and the frame 731 (and/or polymer or other layer 744 lining the inner diameter of the frame 731). In some examples, radial expansion of the inner tube 741 causes expulsion of at least a portion of the blood 745 from one or more openings or channels 739 on the outside of the tube 741 at the outflow end of the device 730.

Without dilation of the native blood vessel, as effected by the frame 731, the space available within the frame for blood flow and expansion of the inner tube/layer 741 would be limited. For example, if the frame 731 had a constant diameter that was only slightly larger than the diameter Dₜ of the tubular channel 749 and/or diameter Dᵥ of the blood vessel 705, the inner tube 741 would be unable to radially expand by a significant or effective amount. Therefore, the ability of the tube 741 to change in volume cyclically in a manner as to add compliance to the blood circulation would be limited. That is, the axially bulging/convex cylindrical shape of the frame 731, wherein the frame has, at its maximum diameter Dₘ, a diameter that is at least 20%, 30%, 40%, and/or 50% greater than the diameter of the native blood vessel, increases the ability of the inner tube 741 to add compliance to the blood circulation.

The outer frame 731 may be at least partially covered with a fabric or polymer coating or cover, which may promote tissue ingrowth with the inner diameter of the native blood vessel and/or provide a fluid-sealing function. The cover may be disposed on an outer surface or area of the frame 731, and/or may be disposed/applied to the inner diameter of the frame 731 on an inside thereof. For example, the layer(s) of covering may be disposed in some implementations between the outer frame 731 and the tube 741. In some implementations, such a cover comprises a cloth or polymer sleeve, which may be at least partially elastic, or alternatively nonelastic. The covering may be applied over or within the frame 731 in any suitable or desirable manner. For example, in some implementations, the cover may be applied using an electrical or mechanical spinning (e.g., rotary jet spinning, electrospinning, or similar) application process or other deposition process known to those having ordinary skill in the art.

The frame 731 may be a self-expandable and/or balloon-expandable stent frame. As described above, the frame 731 may have a bulbous outer diameter, such that the diameter thereof is substantially greater in the axial center than at one or more of the axial ends thereof, as shown. Such bulbous/bulging shape can facilitate tighter fit of the device in the blood vessel in which it is implanted. The frame 731 can include barbs, spikes, or other anchors with an outer diameter thereof configured to engage the target native blood vessel wall and secure the device in place. Such barbs/anchors may puncture through an outer cover/coating and into the native tissue to anchor the frame 731 in place. In some examples, the distal end portion(s) 733 of the frame 731 may have wires or barbs having free ends that may be manipulated to be directed radially outward to puncture the tissue of the native blood vessel 705 and secure the frame 731 in place. In some implementations, such wires/barbs may have shape-memory that predisposes such structure to deflect radially outwardly once deployed from a capsule/sheath to facilitate anchoring of the frame 731 to the native blood vessel.

The compliance conduit 749 formed by the elastic tube 741 is configured to contract and expand as the heart beat cycles, mimicking the compliance of healthy native vascular tissue. The expansion and/or contraction of the inner tube 741 can be facilitated by the changing pressure gradient between the blood in the channel 749 and the blood/medium 745 in the space 703 outside the tube 741. For example, the tube 741 can be biased towards the relaxed cylindrical shape shown in FIG. 13A, such that a decrease in the pressure gradient promotes a return to such state/shape after blood pressure inside the channel 749 causes expansion of the tube 741 (see FIG. 13B), thereby introducing/providing compliance to promote blood flow in the blood vessel 705 (e.g., aorta).

The implant device 730 may or may not include a layer of material on/against the inner diameter of the frame 731. Where such layer is present, axial sealing of the space 703 may be implemented through integration or sealing of the tube 741 with/to the outer layer. Such layer may comprise the same material as the tube 741 and may be an integrated form therewith. For example, the tube 741, together with the outer layer 744 may provide a two-layer implementation with a chamber 703 formed therebetween, which may be similar in some respects to the gas-chamber examples illustrated and described above, except that the chamber(s) 703 may or may not have fluid access opening(s)/channel(s), rather than being fluidly sealed as in the embodiments of FIGS. 6 and 7. The implementation of a layer 744 on the inner diameter of the frame 731 may provide protection against contact with the frame by the inner tube 741, which may cause tearing or other abrasion or damage thereto. The layer(s) 744 may provide a liner for protection or other purposes. For example, the layer can provide fluid-sealing characteristics, which may be desirable in some embodiments to prevent pass-through of fluid from the space 703 out of the device 730 through the frame 731.

In the image of FIG. 13B, the flow channel 749 within the elastic tube 741 is filled with blood/fluid having a higher fluid pressure than that of the blood/medium 745 disposed radially outside of the tube 741 formed around the tubular channel 49. Therefore, as shown, the inner tube 741 is expanded radially outward, thereby reducing the volume of the space 703 between the outer diameter of the tube 741 and the frame 731. Where outflow-end aperture(s)/opening(s) 739 are implemented, fluid/blood disposed within the space 703 is pushed/expelled out of the space 703 and into the circulation downstream of the implant device 730. As the inner diameter 741 expands outward, the volume of the channel 749 increases and energy is stored in the tube 741. For example, energy may be stored in the form of elastic stretch of the material of the inner tube 741. The configuration/state of the implant device 730 in FIG. 13B may be associated with blood pressure conditions associated with the systolic phase of the cardiac cycle.

In the image of FIG. 13A, the stored energy in the tube 741 causes the tube 741 to contract/recoil back towards the original cylindrical tubular state thereof due to decreasing blood/fluid pressure within the channel 749 relative to the pressure in the blood/medium 745 radially outside of the tube 741 in the space 703, thereby providing a secondary pulse in the circulation. That is, as the pressure gradient between the channel 749 and the space 703 decreases, the tube 741 contracts, which returns the stored energy in the tube 741 to the blood circulation. As the tube 741 retracts/returns to the cylindrical shape shown in FIG. 13A, the volume of the channel 749 decreases, which causes the increase in flow through the channel 749. In addition, in some implementations, as the tube contracts, the volume of the space 703 between the tube 741 and the frame 731 increases, which may cause a vacuum effect that draws blood from within the blood vessel 705 into the space 703, as shown. Due to the different fluid dynamics of the blood 745 that is sucked/drawn into the space 703 compared to the blood circulation within the channel 749, the pressure of the blood 745 in the space 103 may be lower than the pressure of the blood within the channel 749 in at least portion(s) of the cardiac cycle, wherein such gradient may advantageously enable the expansion and contraction of the tube 741 to provide the increase in compliance described in detail herein. The configuration/state of the implant device 730 in FIG. 13A may be associated with blood pressure conditions associated with the diastolic phase of the cardiac cycle.

By including openings/accesses 739 into the space 703 outside of the tube 741, the pressure gradient between the space 703 and the channel 749 may be less than certain other examples in which fully-sealed gas chambers are disposed about the flow channel due to the fluid communication between the blood in the channel 749 and the blood 745 in the space 703. Therefore, implementation of sealed gas chambers and compliance restoration devices in accordance with aspects of the present disclosure may be preferable in some instances and/or with respect to certain patients; the implant device 730 of FIGS. 13A, 13B may be implemented with sealed gas chambers on the outer diameter of the tube 741. However, the simplicity of the design of the device 730 shown in FIGS. 13A and 13B with fluid communication between the space 703 and the circulation in the blood vessel 705 can provide certain benefits that may outweigh other considerations. Furthermore, in implementations in which the device 730 does not include sealed gas chambers, risks associated with rupture and/or outgassing from such chamber(s) may be avoided. As referenced above, the device 730 may be implemented with fluid-sealed compressible gas/fluid chamber(s) between the tube 741 and frame 731. The opening(s) 739 into the space 703 around the tube 741 may have any suitable or desirable form more features. For example, some number of sutures or connections between the tube 741 and the frame 731 may be present at one or both ends, wherein gaps between such sutures/connections provide the access opening(s) into and out of the space 703.

The device 730 is shown and described as having opening(s) into the space 703 on the outflow end/side 7330 thereof. However, it should be understood that in some implementations, one or more openings may be present on the inflow side 733i. In such examples, the outflow end 7330 may be sealed off to prevent fluid entry from the outflow end/side, or one or more openings may be present into and out of the space 703 from the inflow 733i and outflow 7330 ends/sides, wherein some amount of blood may flow into the space 703 through the inflow and/or outflow ends and/or may be expelled from the space 703 through one or both ends. In the examples in which the space 703 is sealed on the outflow end 7330 but at least partially open on the inflow end 733i, expulsion from the space of blood/fluid due to expansion of the tube 741 and channel 749 may result in flow of fluid out of the space 703 that moves against the natural flow f of the circulation. In some cases, such upstream flow may be desirable as a mechanism for further controlling/altering the flow of blood in the target blood vessel and/or upstream of the implant 730. In such examples, as the tube 741 contracts/recoils, blood may be drawn into the space 703 from the inflow end 733i, which may further alter flow characteristics of the circulation through the blood vessel.

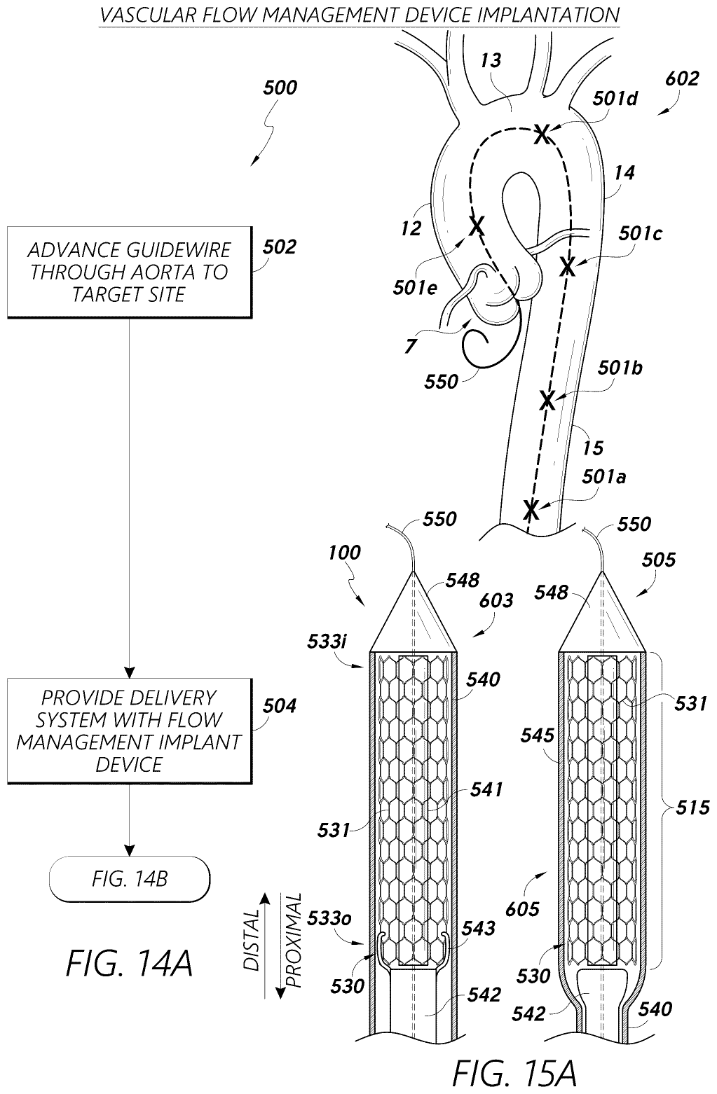

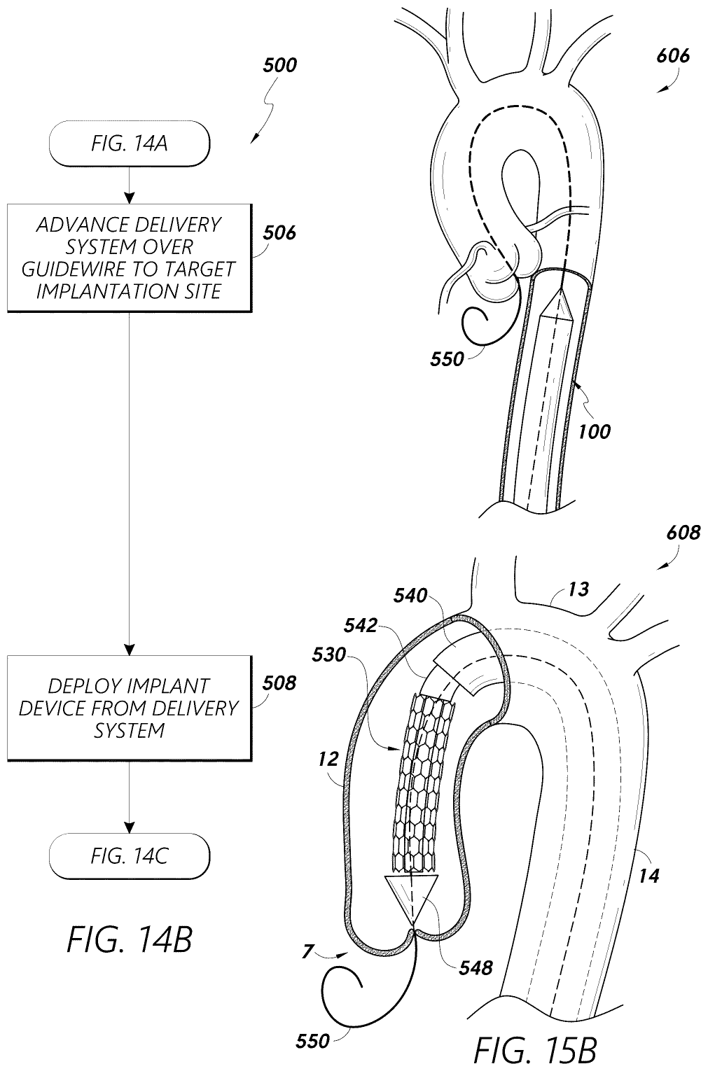

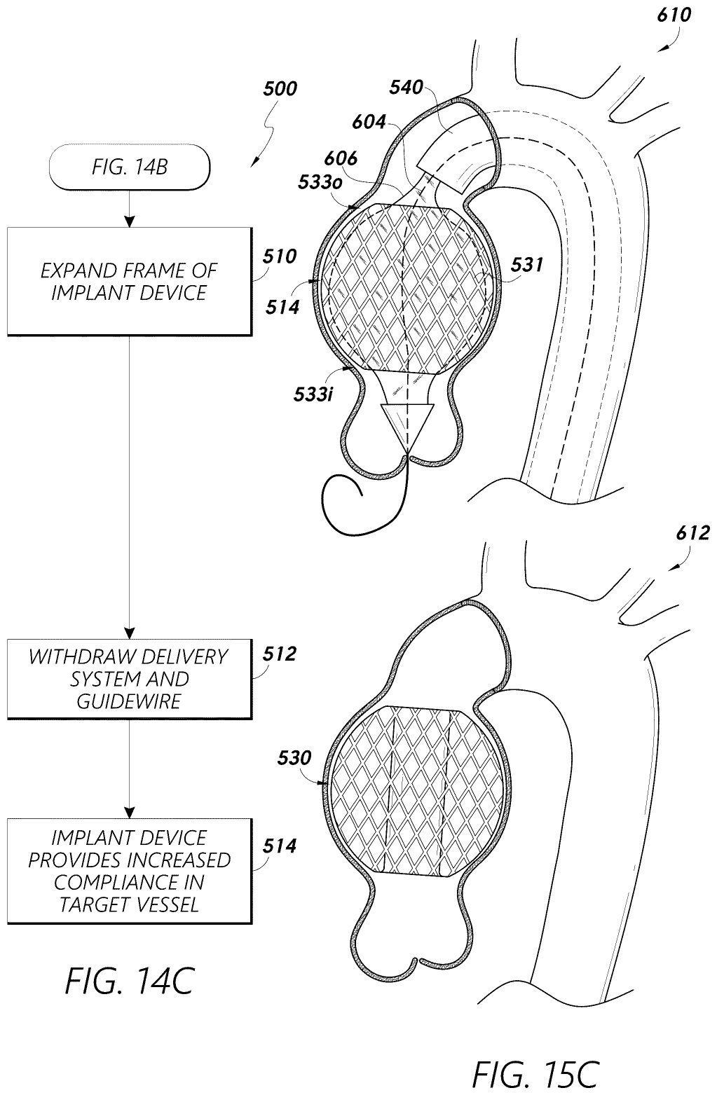

FIGS. 14A, 14B, and 14C illustrate a flow diagram for a process 500 for implanting a compliance implant device 530 in accordance with one or more examples. FIGS. 15A, 15B, and 15C provide images of the compliance implant device 530 and certain anatomy corresponding to operations of the process 500 of FIGS. 14A, 14B, and 14C according to one or more examples. The process 500 utilizes a transcatheter procedure for implantation/deployment of compliance-enhancement implant devices in accordance with aspects of the present disclosure. However, it should be understood that implant devices disclosed herein may be implanted using other types of minimally-invasive and/or surgical procedures.

At block 502, the process 500 involves advancing a guidewire 550 through at least a portion of the aorta 16 of the patient to reach a target implantation site 501. For example, image 602 shows an example implantation site 501a in the abdominal aorta 15, example implantation sites 501b, 501c in the descending thoracic aorta 14, an example implantation site 501d in the aortic arch 13, and an example implantation site 501e in the ascending aorta 12, such as in the area of the aortic valve 7. The guidewire 550 may be advanced through the aortic valve 7, or to any point along the path of the aorta 16. Access to the aorta 16 may be made through any suitable vessel puncture providing access to the arterial system. For example, access may be made via the femoral artery or other arterial blood vessel. In some implementations, access is made to the inferior vena cava via the femoral vein or other access, wherein a guidewire and/or other instrumentation may be crossed over into the abdominal aorta 15 in an area where the inferior vena cava and abdominal aorta 15 are adjacent to one another by puncturing through the venous wall and the arterial wall and advancing through such puncture openings. Although the process 500 and certain other examples are described herein in the context of implantation within the aorta 16, it should be understood that compliance-enhancement devices of the present disclosure may be implanted in other arterial or venous blood vessels, such as the inferior vena cava 19 (see FIG. 1).

Although the process 500 and accompanying illustrations are presented with respect to the implantation of a single compliance-enhancement implant device 530, it should be understood that the process 500 may involve implanting multiple compliance-enhancement implant devices in various positions within the aorta 16 or other blood vessel(s).

At block 504, the process 500 involves providing a delivery system 100 having a compliance-enhancement implant device 530 disposed in a distal portion thereof. Image 603 of FIG. 15A shows a cut-away view of an example implementation of the delivery system 100 in accordance with one or more examples of the present disclosure. The delivery system 100 can comprise one or more catheters or sheaths used to advance and/or implant the compliance-enhancement implant device 530, which may be disposed at least partially within the delivery system 100 during portions of the process 500. The compliance-enhancement implant device 530 can be positioned within the delivery system 100 with a first end thereof (e.g., inflow end 533i) disposed distally and a second end (e.g., outflow end 5330) disposed proximally with respect to the illustrated orientation of the delivery system 100.

In some examples, the delivery system 100 comprises an outer catheter or shaft 540, which may be used to transport the compliance-enhancement implant device 530 to the target implantation site. That is, the compliance-enhancement implant device 530 may be advanced to the target implantation site at least partially within a lumen of the outer shaft 540, such that the compliance-enhancement implant device 530 is held and/or secured at least partially within a distal portion of the outer shaft 540 in a radially compressed configuration.

In some examples, the delivery system 100 comprises a tapered nosecone feature 548, which may facilitate advancement of the distal end of the delivery system 100 through the tortuous anatomy of the patient and/or an outer delivery sheath or other conduit/path. The nosecone 548 may be a separate component from the outer shaft 540 or may be integrated with the outer shaft 540. In some examples, the nosecone 548 is adjacent to and/or integrated with a distal end of the outer shaft 540. In some examples, the nosecone 548 is distally tapered into a generally-conical shape and may comprise and/or be formed of multiple flap-type forms that can be urged/spread apart when the compliance-enhancement implant device 530 and/or any portions thereof, interior shafts, or devices, are advanced distally therethrough.

The delivery system 100 may further be configured to have the guidewire 550 disposed at least partially within the delivery system 100 and/or coupled thereto in a manner to allow the delivery system 100 to follow a path defined by the guidewire 550. In some implementations, the guidewire 550 may pass through an interior of the implant device 530 and/or through a lumen of a pusher device or tube 542 of the delivery system 100.

The compliance-enhancement implant device 530 may have any of the features of any one or more of the examples described in detail herein, including an outer frame 531 and an inner radially expandable tube 541, which may or may not include compressible-gas-filled chamber(s) disposed thereabout, and may be at least partially sealed to the frame 531 at one or both axial ends thereof. The implant device 530 may be disposed within the shaft/sheath 540 in a radially compressed/collapsed configuration, wherein the frame 531 and/or tube 541 is/are crimped to assume a reduce radial profile. In the compressed delivery configuration, the device 530 may be somewhat elongated compared to a fully-expanded configuration thereof due to at least some of the struts/cells of the frame 531 being deflected into more longitudinally-oriented configurations when radially crimped/compressed.

The delivery system 100 may optionally comprise the illustrated pusher shaft 542, which may be slidingly disposed within the outer sheath 540 proximal and/or adjacent to the implant device 530. The pusher 542 can be configured to be used to push/advance the frame 531 and/or other component(s) of the implant device 530 relative to the outer shaft/sheath 540 as a means to deploy the device 530 from the sheath 540. For example, the pusher 542 may be distally advanced relative to the outer sheath 540 to cause distal advancement of the compliance-enhancement implant device 530 through a distal opening in the outer sheath/shaft 540. Alternatively (or additionally), the implant device 530 may be deployed from the outer sheath 540 at least in part by proximally pulling the outer sheath 540 relative to the pusher 542.

In some examples, the pusher 542 is releasably attached to the frame 531 and/or other component(s) of the implant device 530, wherein after the device 530 has been deployed from the sheath 540, positioned in the desired implantation site/position, and/or expanded, the pusher 542 (or other component of the delivery system 100) may be disengaged from the implant device 530 to release the device 530 and allow for removal/withdrawal of the delivery system 100. For example, the pusher 542 or other component(s) of the delivery system 100 may comprise one or more feet or arms that project distally and/or radially from the pusher. In some implementations, to deploy the implant device 530, the outer sheath 540 is proximally pulled and/or the pusher 542 is distally pushed to thereby draw the sheath 540 past the distal end 533i of the implant device 530, at least partially exposing/deploying the frame 531 and implant device 530. Initially, the sheath 540 may only be withdrawn to a position that exposes a portion of the frame 531 and/or other portion(s) of the device 530, while maintaining position around and holding coupling arms associated with the pusher 542, wherein further withdrawal of the sheath 540 is implemented after the position of the implant device 530 in the anatomy has been confirmed, wherein withdrawing the sheath 540 proximally past the coupling arms of the pusher 542 causes the arms to radially deflect, thereby disengaging from the frame 531 and/or device 530 in a manner as to release the device. In some examples, no such coupling arms are associated with the pusher 542. The implant device 530 may comprise one or more radiopaque markers that may be referenced to determine/confirm the position of the implant device 530 at various stage(s) of the process 500 using a suitable imaging modality.

The image 605 shows an alternative delivery system 505, which includes a distal capsule portion 515, wherein the implant device 530 is disposed in the compressed configuration within the capsule portion 515. Deployment of the implant device 530 may be implemented at least in part by proximally pulling the capsule sheath 545 such that the implant device 530 is pressed proximally against the pusher 542 to keep the implant 530 in place while unsheathing is taking place. In some implementations, the capsule portion 515 may have a diameter that is greater than that of the sheath 540 in the area proximal to the capsule 515.

At block 506, the process 500 involves advancing the delivery system 100 over the guidewire 550 until the target implantation site is reached to thereby position the implant device 530 for deployment in the target anatomy. At block 508, the process 500 involves deploying the implant device 530 from the delivery system 100, which may be performed in any of the manners described above. Once the device 530 is deployed from the delivery system 100, the process 500, at block 510, may involve expanding the frame 531 and/or tube 541 of the implant device 530 to thereby secure the implant 530 in place in the deployed/expanded configuration thereof. The expansion operation(s) associated with block 508 may further involve dilating the native blood vessel by expanding the frame 531 to a diameter, at least with respect to a lengthwise portion thereof, that is substantially greater (e.g., more than 20-30% greater) than the diameter of the native blood vessel, wherein such dilation of the blood vessel may serve to form/introduce a space for radial expansion of the complaint tube 541 and/or one or more portions/layers thereof, as described in detail herein.

The device 530 may be mounted on a balloon 606 of a delivery shaft 604. The device 530 can be situated on the balloon 606 such that an inflow end 533i is disposed distally on the balloon 606 and an outflow end 5330 is disposed proximally on the balloon 606. When the balloon 606 is inflated, the frame 531 expands around the balloon 606. The balloon 606 may serve to expand the struts of the frame 531 by expanding the inner tube 541 within the frame 531 to push outwardly against the frame 531. In examples in which the inner tube 541 is part of a tubular member comprising compressible, gas-filled chamber(s), as described in detail above, the gas in the chamber(s) may compress to some degree during expansion of the balloon 606, but also exert outward radial force on the frame 531 to cause expansion thereof. In examples that do not comprise gas-filled chamber(s) outside of the inner tube 541, the inner tube 541 may expand and press against the inner diameter of the frame 531 to expand the frame. A layer of coating, cloth, or other covering/layer may be disposed between the inner tube 541 and the frame 531, wherein such layer may line the inner diameter of the frame 531.

In some implementations, the frame 531 may be expanded/dilated using pull wire(s) that are configured to be pulled or pushed to cause the frame to expand to the expanded configuration shown. For example, pull wire(s) may be coupled to the distal portion of the frame 531 such that pulling the wire(s) proximally causes the ends of the frame 531 to be brought together, thereby dilating/expanding the frame 531. In some implementations, expansion of the frame 531 may be achieved via shape memory features of the frame 531. For example, the frame 531 may comprise nitinol or other shape-memory metal configured to self-expand when released from the delivery sheath/capsule.

The balloon 606 may be configured to expand in an axially bowed-out, rounded/spherical shape conforming to the convex/bowed cylindrical shape of the frame 531. After the balloon 606 has expanded the frame 531 to the convex/bowed cylindrical shape to dilate the blood vessel, the balloon 606 can be deflated and removed. Controlling the position of the compliance implant device 530 on the balloon 606 can be important during delivery in view of the longitudinal shortening of the frame 531 that may occur during expansion, which can cause the device 530 to move relative to the balloon 606. In some implementations, an intermediate portion 514 of the device/frame 531, which may be configured to expand to the widest diameter of the lengthwise portions of the frame 531, can be held constant relative to the balloon, while the inflow and outflow end portions 533 may foreshorten to some degree towards the intermediate portion 514 during expansion.

At block 512, the process 500 involves withdrawing the delivery system 100 and guidewire 550, leaving the implant device 530 implanted in the aorta or other target blood vessel, as shown in image 610. With the implant device 530 implanted, as shown, the increasing compliance provided by the implant device 530 can improve arterial blood flow and/or prevent elevated blood pressure. Other benefits may also be achieved, as described in detail herein.

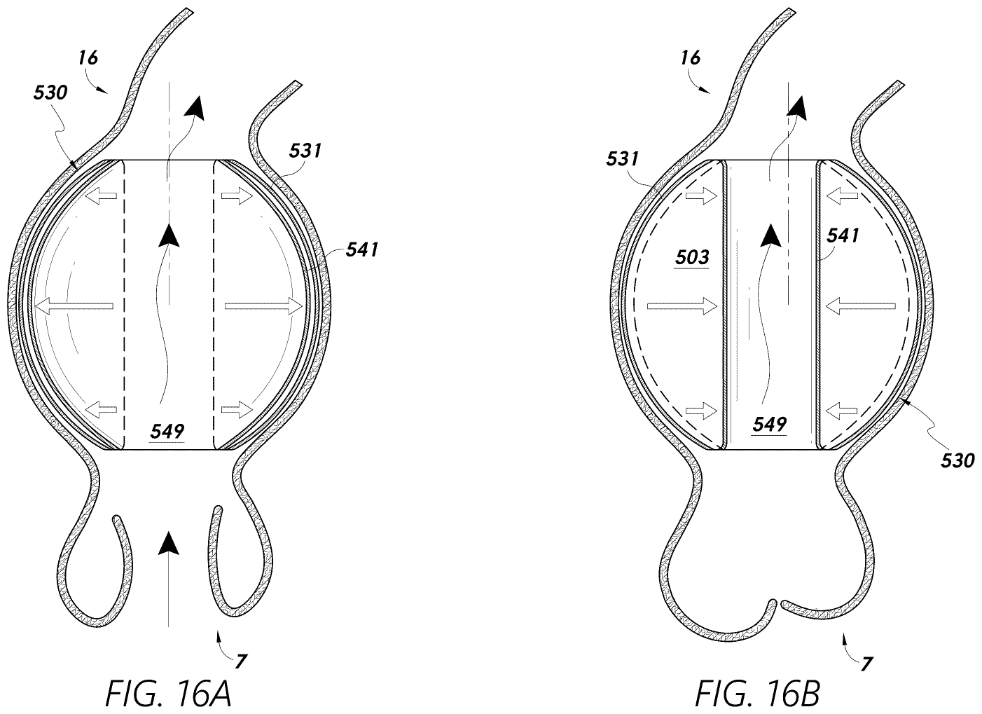

FIGS. 16A and 16B show the compliance-enhancement action of the implant device 530 described above during high-pressure (e.g., systole) and low-pressure (e.g., diastole) phases/conditions, respectively. As shown in FIG. 16A, when blood is received within the channel 549 of the expandable tube 541 that has pressure that is greater than the pressure immediately outside of the tube 541, the tube/layer 541 may expand into the space 503 provided outside of the tube 541 due to the expansion of the frame 531 and dilation of the blood vessel. Such expansion into the space 503 results in stored energy in the tube 541 and/or in gas compressed outside of the tube 541 due to the expansion of the tube 541. That is, the elastic recoil characteristics of the tube 541 and/or the expansion energy of compressed gas (not shown in FIGS. 16A, 16B) disposed about the tube 541 can increase in response to the radial expansion of the tube 541. The expansion of the tube 541 serves to at least partially increase the volume of, and reduce the pressure in, the channel 549 during the high-pressure phase.

As demonstrated in FIG. 16B, when the pressure within the channel 549 reduces relative to the pressure radially outside of the tube 541, the stored energy in the tube 541 and/or compressed gas may be returned to the blood circulation by reducing the volume of the channel 549 and increasing to some degree the pressure therein, thereby pushing blood through the channel 549 and out the outflow end 5330 of the device 530. Such volume reduction and pressure increase occurs in response to recoil/contraction of the tube 541 to the cylindrical shape/form shown.

### Valve-Integrated Compliance-Enhancement Implants

In some implementations, examples of the present disclosure provide implants that include compliance-enhancing radially-expandable tubes, as well as prosthetic valve features, wherein such devices are configured to function as blood vessel flow optimizing/increasing devices configured to generate vascular compliance, and may further provide a replacement for a diseased heart valve (e.g., aortic valve) and/or serve to otherwise advantageously provide one-way flow control.

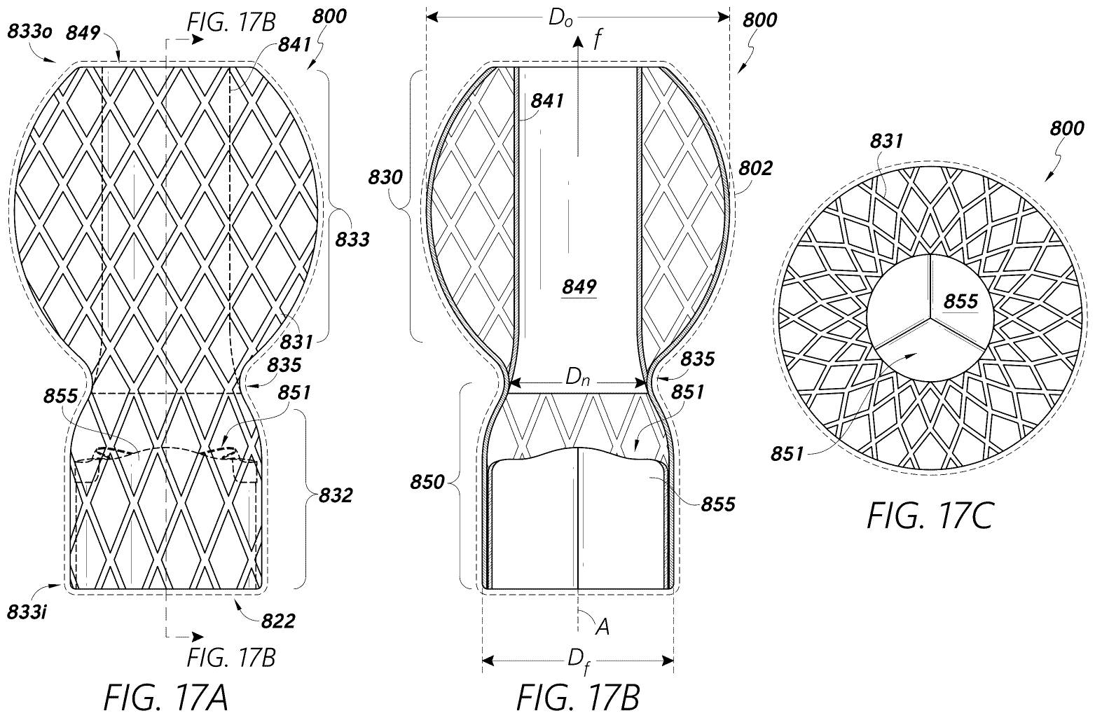

FIGS. 17A-17C provide side, side cross-sectional, and axial views, respectively of a valved compliance implant device 800 in accordance with one or more examples. The device 800 can be utilized as an arterial flow optimizer. The device 800 can include a compliance-enhancement segment/portion 830, which may be axially offset from a valved segment/portion 850, wherein the compliance-enhancement segment 830 and the valved segment 850 work together to control blood flow through the device 800. In some examples, the valved segment 850 may be used to replace a diseased aortic valve or other heart valve.

The device 830 includes a frame 831, which includes a first portion 832 associated with the valved segment 850 and a second portion 833 associated with the compliance-enhancement segment 830, wherein either or both of the frame portions 832, 833 may be balloon-expandable and/or self-expandable. In some implementations, the implant device 800 may be placed in the ascending aorta, with a prosthetic valve 851 of the valved segment 850 aligned with the native aortic valve. However, it should be understood that the implant device 800 can be placed/implanted anywhere in a patient's vasculature, such as within the descending aorta, thoracic aorta, inferior vena cava, superior vena cava, or other blood vessel. As implanted and maintained within the target blood vessel, the device 800 can improve arterial blood flow and/or prevent elevated blood pressure. The implant device 800 may be delivered using a minimally-invasive approach, such as using a catheter. Delivery and/or implantation of the device 800 may be guided by monitoring one or more radiopaque markers on the implant device 800.

The implant device 800 can provide multiple benefits and/or solutions for the patient. For example, the device 800 can provide a replacement of a diseased native valve, as well as introduce a vessel compliance-enhancing mechanism into the blood circulation. The device 800 may provide a low-profile, collapsible/self-expandable structure, which may be deliverable in a radially-compressed/collapsed state within a catheter/sheath, as described in detail above. The frame 831 may comprise any suitable or desirable material or form, as described in detail herein in connection with any of the examples of the present disclosure, such as stainless steel, cobalt-chromium alloy, or nickel-titanium alloy (e.g., nitinol). Use of shape-memory material for the frame 831 may allow for self-expansion of the device 800, as with other examples disclosed herein.

The compliance-enhancement segment 830 may comprise an inner, radially-expanding tube 841, which may or may not have compressible-gas-filled chamber(s) disposed thereabout. The prosthetic valve segment 850 may comprise valve leaflets 855 configured to control flow through the device 800, such as in a one-way valve configuration. The tube 841 and leaflets 855 may comprise any suitable or desirable material(s), such as biological tissue or polymer materials; the tube 841 and leaflets 855 may comprise a common material, or may comprise different materials. Therefore, the compliance-enhancement segment 830 of the device 800 may be understood to be similar or identical to the device 30 of FIGS. 6A-6E, the device 130 of FIGS. 8A-8E, the device 730 of FIGS. 13A and 13B, or any other example device described herein, wherein such device forms an axial segment/portion of the device 800.

The compliance-enhancement segment 830 may be configured with the shape, form, structure, and/or function of any of the compliance-enhancement devices disclosed herein. For example, a compliance enhancement device of any of the examples disclosed herein may be implemented as part of the device 800, wherein a frame of the compliance-enhancement device is coupled to or integrated with the frame portion 832 in an axial/series arrangement.

The device 830 includes a frame portion 835 that joins the compliance-enhancement segment 830 with the valved segment 850. The frame portion 835 may be considered a neck portion of the frame 831 and may have a diameter Dₙ that is less than either or both of the diameters Dₒ, D_{f} of the compliance enhancement segment 833 and the valved segment 832, respectively. Therefore, the frame 831 may, in some implementations, have an hourglass-type shape from the side perspective shown in FIG. 17A with radially bulging segments 832, 833 separated by a narrower axial neck segment 835. In some examples, the length of the device 800 may be between 40-60 mm. For example, the compliance-enhancement segment 830 may have a length of approximately 20-40 mm, such as about 30 mm, whereas the valved segment 850 may have a length of between 10-30 mm, such as about 20 mm. Furthermore, the neck portion 835 may be between 0-10 mm in length, wherein the combined length of the various segments of the frame/device can be about 50 mm, or other value between 40-60 mm.

Although FIGS. 17A-17C show an example of the device 800 in which the flow-control/valved portion 850 is disposed at an inflow side/end 833i of the device 800, whereas the compliance-enhancement portion 830 is disposed at an outflow side/end 8330 of the device 800, it should be understood that the flow-control portion 850 may be positioned at/adjacent the outflow side/end 8330 and the compliance-enhancement portion 830 may be positioned at/adjacent the inflow side/end 833i, depending on the particular example.

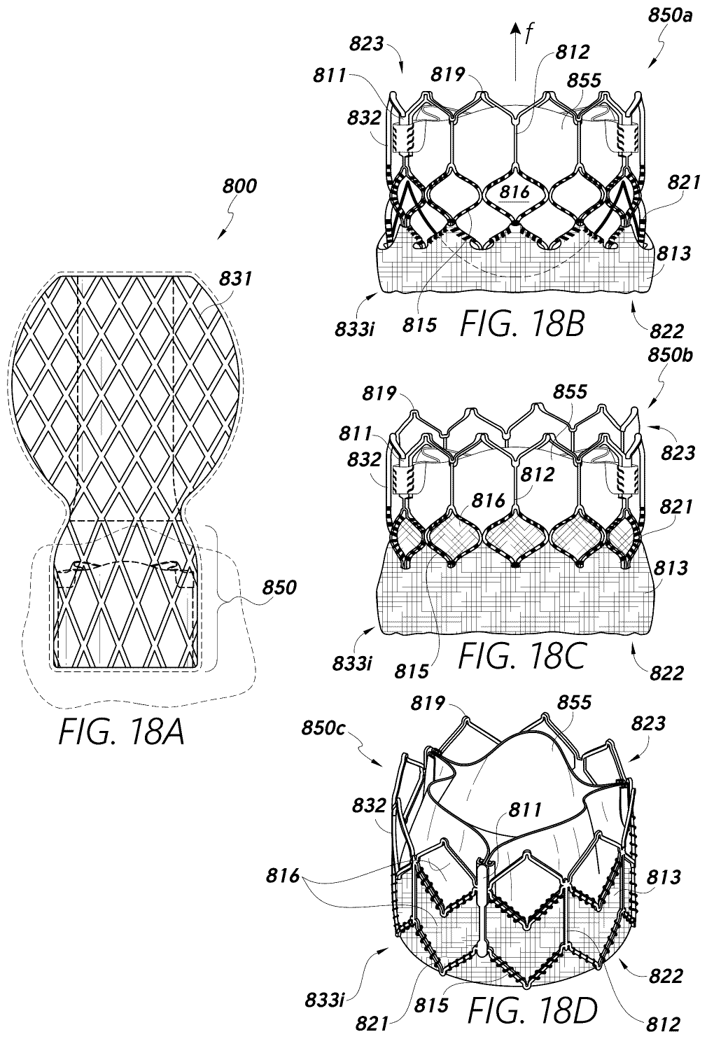

FIG. 18A shows the compliance-enhancing implant device 800 of FIGS. 17A-17C, which includes a valve portion 850 in accordance with one or more examples.

FIGS. 18B, 18C, and 18D show respective designs of example implementations of the valved portion 850 of the compliance implant device 800 in accordance with one or more examples. The valved portion 850 is shown as including valve leaflets 851. However, it should be understood that the portion 850 may include any other type of flow-control mechanisms that may be utilized within the frame segment 832 to achieve a desired direction and/or rate of flow through the device 800 in accordance with aspects of the present disclosure. The compliance-enhancement implant device 800 is shown in FIGS. 18B-18D as including one or more one-way valves 851, which may be configured and/or oriented to permit flow in a desired flow direction f through the channel 849 of the device 800, while restricting or blocking flow in the reverse direction.

The valve feature(s) 855 associated with compliance-enhancement implant devices in accordance with aspects of the present disclosure may function by opening to permit flow in the presence of a pressure gradient in the direction of the valve, such that the valves open to permit flow and close with each cardiac cycle to prevent flow in the opposite direction.

As described above, the frame 831, including the valved segment 832, can be designed to be radially crimped or compressed to facilitate endovascular delivery to the target implant site. For example, the valved portion 850 may be positioned at a native valve annulus (e.g., aortic valve annulus), where the frame 831 may be expanded to an operational state, for example, by an expansion balloon, such that the leaflet structure 851 or other flow-control mechanism of the valved portion 850 regulates blood flow through the native valve annulus. The frame 831 can be made of nitinol or another self-expanding material. In some implementations, the valved segment 832 of the frame 831 can be plastically expandable to its functional size by a balloon or another expansion device, in which case the frame can be made of a plastically expandable material, such as stainless steel or a cobalt chromium alloy. Other suitable materials can also be used.

In some implementations, the device 800 can be mechanically expanded or radially self-expand from a compressed delivery state to the operational state under its own resiliency when released from a delivery sheath. The following description of the valved portion/segment 850 of the device 800 may be understood with respect to any of the examples of FIGS. 18B-18D. The example implementations of the valved portion 850 of the device 800 shown of FIGS. 18B, 18C, and/or 18D may be similar in one or more respects to the Edwards Lifesciences SAPIEN XT™, SAPIEN 3™, and/or SAPIEN 3 Ultra™ transcatheter prosthetic heart valves.

The valved portion 850 may comprise an inflow end 822 and an outflow end 823, which may lead into the neck portion 835 of the device 800. The valved portion 850 includes a segment 832 of the frame 831 of the device 800. The valved portion 850 may further comprise a leaflet structure 855 supported inside the frame segment 832. In some implementations, the frame segment 832 is at least partially covered within and/or without by a sealing skirt 813, which may comprise any suitable or desirable material, such as textured polyethylene terephthalate (PET). The skirt 813 can be attached to an inner surface of the frame 832 to form a suitable/desirable attachment surface for the valve leaflets 855 of the valve 851.

The frame segment 832 can comprise an annular structure having a plurality of vertically extending commissure attachment posts 811, which attach and help shape the leaflet structure 855 therein. Additional vertical posts or strut members 812, along with circumferentially extending strut members 815, help form the rest of the frame 832. In some examples, the struts 815 and/or 812 may form axial rows of cells 816, which may be circumferentially staggered/offset in the valve segment 832 (and/or the rest of the frame 831) as in the examples of FIGS. 18B and 18C, or the rows of cells 816 may be generally circumferentially aligned, as in the example of FIG. 18D. The open cell geometry of the frame 831 can facilitate coronary access. With further reference to the example of FIG. 18D, the strut members 815 of the frame 832 may form chevron/zig-zag shapes/cells in a base portion of the frame segment 832. The struts 815 may form edged crown portions 819 or apices at the inflow and/or outflow ends of the valved segment 832 of the frame 831.

The skirt 813 can be attached to the frame segment 832 in any suitable or desirable manner, such as through the use of adhesive or other attachment means. In some examples, the skirt 813 is attached to an inner and/or outer surface of the valve frame 832 via one or more sutures 821, which may be wrapped around the various struts of the frame segment 832, as needed. The skirt 813 may provide a relatively more substantive attachment surface for portions of the leaflet structure 855 positioned closer to the inflow end 822 of the device 800. In some examples, as shown in FIGS. 18B and 18C, the skirt 813 may be folded over the inflow end of the frame 832 to cover over an outer diameter/surface of the frame on the inflow end portion 833i of the device, wherein the portion of the skirt 813 on the outside of the frame 832 may facilitate attachment to the patient anatomy, such as through tissue ingrowth and/or frictional fit between the frame 832 and the anatomy (e.g., aortic valve annulus, aorta, etc.). The valve 855 may comprise any suitable or desirable number of leaflets, such as three, as shown in the illustrated examples.

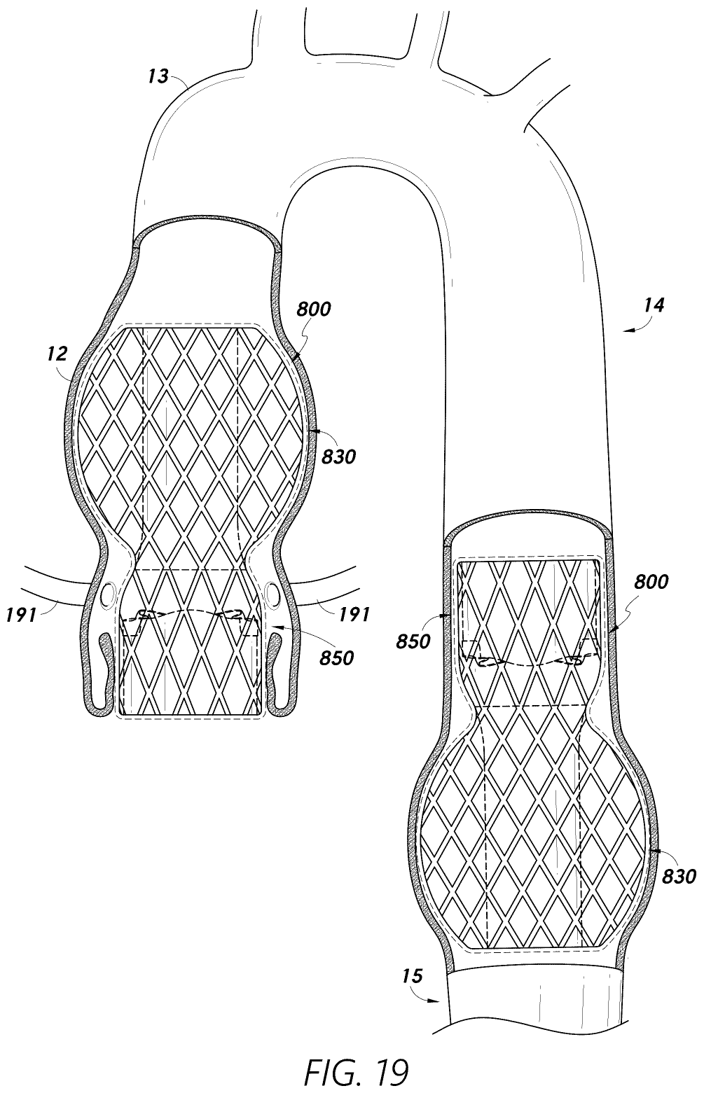

FIG. 19 shows aortic anatomy with valved compliance implant devices deployed in example positions in the anatomy in accordance with one or more examples. The hourglass shape of the device 800 and/or frame 831 may facilitate secure lodging/securement within the target anatomy, which may be, for example, an ascending portion of the aorta, or other blood vessel segment. For example, the narrower neck portion 835 may allow for the frame 831 to bend/curve to some degree to conform to the target anatomy (e.g., aorta), Furthermore, the wider valved segment 832 may be sized to fit within the relatively wider space of the ascending aorta in the area immediately adjacent or within the aortic valve annulus and/or in the area where the coronary arteries 191 emanate from the aorta. In some implementations, the valved portion 850 is configured to be positioned and secured within the native aortic valve annulus, and the expanded diameter of the frame segment 832 may accommodate such positioning.

The frame 831 may be a self-expandable and/or balloon-assisted stent with an elastic compliance-enhancement member as well as a prosthetic heart valve, wherein the device 800 is configured to be placed in the ascending aorta 12, with the prosthetic valve aligned with the diseased native aortic valve 7. Although shown implanted in and/or near to the aortic valve 7, it should be understood that the device 800 can be placed anywhere within a patient's vasculature. The implant device 800 may serve to improve arterial blood flow and/or prevent elevated blood pressure. The frame 831 may be a one-piece frame having an hour-glass shape, as shown. That is, the frame segments 832, 833 may be integrated in a common frame/form, wherein a neck portion 835 divides the segments 832, 833 of the frame. Although shown in some examples as being axially outwardly bulging it should be understood that frame segments of the device 800 may not be bulging, but rather may be straight, as shown in the examples of FIGS. 18B, 18C, and 18D. That is, the diameter of the valve segment 832 and/or compliance segment 833 may be continuous over at least a length/portion thereof.

The device 800 may be delivered to the target anatomy using a minimally-invasive approach, such as using a process involving a catheter guided by a radiopaque marker associated with the implant 800. In some implementations, the device 800 can be implanted transapically through minimally invasive access to the apical region of the ventricle(s) of the heart and through the ventricle(s) to the target aortic anatomy. In some implementations, the native aortic valve 7 is left intact, wherein the valved portion 850 of the device 800 provides an additional valve in the ascending and/or descending portion(s) of the aorta 16. In some implementations, the device 800 may be implanted on either side of the renal arteries, where in the valved portion 850 may serve to prevent backflow and/or help with perfusion of the kidneys. For example, the device 800 may serve to increase the pressure on the arterial side of the kidney circulation, thereby improving perfusion thereof.

### Sterilization

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### Additional Description of Examples

Provided below is a list of examples, each of which may include aspects of any of the other examples disclosed herein. Furthermore, aspects of any example described above may be implemented in any of the numbered examples provided below.

Example 1: An implant device comprising a frame having an axially bulging cylindrical form in an expanded configuration of the frame, and a radially-expandable tube disposed within the frame.

Example 2: The implant device of any example herein, in particular example 1, wherein a medial diameter of the frame is at least 20% greater than an end diameter of the frame at at least one axial end of the frame.

Example 3: The implant device of any example herein, in particular example 2, wherein the frame expands from the end diameter to the medial diameter in outer quartile portions of a length of the frame, such that the frame has the medial diameter over at least half of the length of the frame.

Example 4: The implant device of any example herein, in particular example 2 or example 3, wherein a diameter of the frame expands gradually from the end diameter to the medial diameter to form a convex cylinder shape.

Example 5: The implant device of any example herein, in particular any of examples 1-4, wherein the tube, in a relaxed configuration thereof, has a diameter that is at least 20% less than a medial diameter of the frame.

Example 6: The implant device of any example herein, in particular example 5, wherein the diameter of the tube is within 10% of an end diameter of the frame.

Example 7: The implant device of any example herein, in particular any of examples 1-6, wherein the tube is coupled to the frame at first and second axial ends of the implant device.

Example 8: The implant device of any example herein, in particular example 7, wherein one or more openings are present at the first axial end of the implant device, the one or more openings providing one or more flow channels into a space between an outer diameter of the tube and an inner diameter of the frame.

Example 9: The implant device of any example herein, in particular example 8, wherein the one or more openings comprise one or more gaps between the tube and the frame at the first axial end of the implant device.

Example 10: The implant device of any example herein, in particular example 9, wherein an axial end of the tube is coupled to an axial end of the frame with suturing, and the one or more openings are formed by one or more gaps in the suturing.

Example 11: The implant device of any example herein, in particular example 10, wherein the axial end of the tube deflects radially inward in at least one of the one or more gaps in the suturing to form one or more inlet channels into the space between the outer diameter of the tube and the inner diameter of the frame.

Example 12: The implant device of any example herein, in particular any of examples 1-11, further comprising one or more fluid-filled chambers disposed between the radially-expandable tube and the frame.

Example 13: The implant device of any example herein, in particular example 12, wherein the one or more fluid-filled chambers have compressible gas contained therein.

Example 14: The implant device of any example herein, in particular example 13, wherein the one or more fluid-filled chambers have foam disposed therein.

Example 15: The implant device of any example herein, in particular any of examples 1-14, wherein the tube is coupled at first and second axial ends thereof to respective first and second axial ends of the frame by at least one of heat-sealing, machine-crimping, or suturing.

Example 16: The implant device of any example herein, in particular any of examples 1-15, wherein the tube is sutured to a suture ring associated with an axial end of the frame.

Example 17: The implant device of any example herein, in particular any of examples 1-16, wherein the radially-expandable tube is disposed within a first axial portion of the frame, the axially bulging cylindrical form being associated with the first axial portion, and the frame includes a second axial portion having a valve associated therewith.

Example 18: The implant device of any example herein, in particular example 17, wherein the frame further includes a neck portion disposed between the first axial portion and the second axial portion.

Example 19: The implant device of any example herein, in particular example 18, wherein the neck portion of the frame has a diameter that is less than a both a maximum diameter of the first axial portion and a maximum diameter of the second axial portion, forming an hourglass shape of the frame.

Example 20: The implant device of any example herein, in particular example 19, wherein the second axial portion has a sealing skirt sutured thereto.

Example 21: The implant device of any example herein, in particular example 20, wherein the sealing skirt is wrapped around an axial end of the frame such that the sealing skirt is disposed on both an outer surface and an inner surface of the second axial portion of the frame.

Example 22: The implant device of any example herein, in particular example 17, wherein the valve comprises a plurality of leaflets coupled to the frame in the second axial portion of the frame.

Example 23: The implant device of any example herein, in particular example 17, further comprising one or more gas-filled chambers disposed between the radially-expandable tube and the frame in the first axial portion of the frame.

Example 24: The implant device of any example herein, in particular example 17, wherein the first axial portion is downstream of the second axial portion relative to a flow direction associated with the valve.

Example 25: An implant device comprising a frame having an axially-convex form that expands in diameter moving from first and second axial ends of the frame to a medial portion of the frame, and a cylindrical toroid balloon member disposed at least partially within the frame, the balloon member forming a central axial flow channel.

Example 26: The implant device of any example herein, in particular example 25, wherein the balloon member comprises one or more gas-filled chambers.

Example 27: The implant device of any example herein, in particular example 26, wherein the one or more gas-filled chambers are formed between an outer layer of the balloon member and an inner layer of the balloon member that forms the central axial flow channel.

Example 28: The implant device of any example herein, in particular any of examples 26 or 27, wherein the one or more gas-filled chambers occupy a space between the flow channel and the frame.

Example 29: The implant device of any example herein, in particular any of examples 26-28, wherein the one or more gas-filled chambers are configured to radially compress when a pressure within the flow channel is greater than a pressure of gas within the one or more gas-filled chambers.

Example 30: The implant device of any example herein, in particular example 29, wherein the frame includes radially-outwardly-projecting tissue anchors configured to embed in a wall of a blood vessel when the frame is expanded within the blood vessel.

Example 31: The implant device of any example herein, in particular any of examples 26-30, wherein a medial diameter of the frame is at least 20% greater than an end diameter of the frame at at least one axial end of the frame.

Example 32: The implant device of any example herein, in particular example 31, wherein a diameter of the frame expands gradually from the end diameter to the medial diameter to form a convex cylinder shape.

Example 33: The implant device of any example herein, in particular any of examples 26-32, wherein the flow channel, when the balloon member is in a non-compressed configuration, has a diameter that is at least 20% less than a medial diameter of the frame.

Example 34: The implant device of any example herein, in particular example 33, wherein the diameter of the flow channel is within 10% of an end diameter of the frame when the balloon member is in the non-compressed configuration.

Example 35: An implant device comprising a tubular frame comprising a first axial segment and a second axial segment, a radially-expandable tube disposed within the first axial segment of the frame, and a one-way valve disposed within the second axial segment of the frame.

Example 36: The implant device of any example herein, in particular example 35, wherein the first axial segment has a first maximum diameter, the second axial segment has a second maximum diameter, and a third axial segment of the frame disposed between the first axial segment has a third maximum diameter that is less than the first maximum diameter and the second maximum diameter.

Example 37: The implant device of any example herein, in particular any of examples 35 or 36, wherein the first axial segment has an axially bulging form that increases in diameter from a first diameter at an axial end of the first axial segment to a second diameter in a medial axial portion of the first axial segment that is at least 20% greater than the first diameter.

Example 38: The implant device of any example herein, in particular example 37, wherein, when the radially-expandable tube is in a non-expanded configuration, a space is present between an outer diameter of the tube and an inner diameter of the frame, the space provide a volume into which the tube can radially expand.

Example 39: The implant device of any example herein, in particular example 38, wherein the tube is coupled to the first axial segment in a manner as to provide fluid access into the space between the outer diameter of the tube and the inner diameter of the frame.

Example 40: The implant device of any example herein, in particular any of examples 38 or 39, wherein the tube comprises one or more holes providing fluid access into the space between the outer diameter of the tube and the inner diameter of the frame.

Example 41: A method of controlling blood flow in a blood vessel, the method comprising providing a delivery system having disposed therein an implant device, the implant device comprising a tubular frame in a radially-crimped configuration, and a radially-expandable tube disposed within the frame. The method further comprises advancing a distal portion of the delivery system to a target position within a portion of an aorta of a patient, deploying the implant device from a distal portion of the delivery system, and dilating the portion of the aorta by radially expanding the frame to an axially bulging cylindrical form having a medial diameter that is at least 20% greater than a diameter of the portion of the aorta prior to said dilating.

Example 42: The method of any example herein, in particular example 41, further comprising withdrawing the delivery system from the aorta, and receiving blood flow within a flow channel formed within the tube when the tube is in a non-expanded configuration, said blood flow causing the tube to radially expand into a volume between the flow channel and the axially bulging cylindrical form of the frame.

Example 43: The method of any example herein, in particular example 42, wherein, after said radially expanding of the tube, the tube radially contracts, thereby pushing blood through the implant device.

Example 44: The method of any example herein, in particular example 43, wherein said radial contraction of the tube causes blood to be drawn into the volume in a space between an outer surface of the tube and an interior surface of the frame.

Example 45: The method of any example herein, in particular example 44, wherein, after said drawing of blood into the space between the outer surface of the tube and the interior surface of the frame, fluid pressure within the flow channel is greater than fluid pressure in the space.

Example 46: The method of any example herein, in particular any of examples 44 or 45, wherein the tube is coupled to the frame at first and second axial ends of the implant device.

Example 47: The method of any example herein, in particular example 46, wherein one or more openings are present at the first axial end of the implant device, and said drawing of blood into the space between the outer surface of the tube and the interior surface of the frame is through the one or more openings.

Example 48: The method of any example herein, in particular any of examples 41-47, wherein the medial diameter is at least 30% greater than the diameter of the portion of the aorta.

Example 49: The method of any example herein, in particular any of examples 41-48, wherein the medial diameter is at least 40% greater than the diameter of the portion of the aorta.

Example 50: The method of any example herein, in particular any of examples 41-49, wherein the axially bulging cylindrical form has a diameter that expands gradually from end portions thereof to the medial diameter at an axial center of the frame.

Example 51: The method of any example herein, in particular any of examples 41-50, wherein, after said radially expanding the frame, an end diameter of the frame is within 10% of the diameter of the portion of the aorta.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s), Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An implant device for restoring compliance to a blood vessel, the implant device comprising:
a frame having an axially bulging cylindrical form in an expanded configuration of the frame; and
a radially-expandable elastic polymer tube disposed within the frame, the tube being attached to a first axial end of the frame and extending a length of the frame between the first axial end and a second axial end.

2. The implant device of claim 1, wherein a medial diameter of the frame is at least 20% greater than an end diameter of the frame at at least one axial end of the frame, wherein:
the frame expands from the end diameter to the medial diameter in outer quartile portions of a length of the frame, such that the frame has the medial diameter over at least half of the length of the frame; or
a diameter of the frame expands gradually from the end diameter to the medial diameter to form a convex cylinder shape.

3. The implant device of claim 1, wherein the tube, in a relaxed configuration thereof, has a diameter that is at least 20% less than a medial diameter of the frame.

4. The implant device of claim 1, wherein the tube is coupled to the frame at first and second axial ends of the implant device.

5. The implant device of claim 4, wherein one or more openings are present at the first axial end of the implant device, the one or more openings providing one or more flow channels into a space between an outer diameter of the tube and an inner diameter of the frame.

6. The implant device of claim 5, wherein:
an axial end of the tube is coupled to an axial end of the frame with suturing; and
the one or more openings are formed by one or more gaps in the suturing.

7. The implant device of claim 6, wherein the axial end of the tube deflects radially inward in at least one of the one or more gaps in the suturing to form one or more inlet channels into the space between the outer diameter of the tube and the inner diameter of the frame.

8. The implant device of claim 1, further comprising one or more fluid-filled chambers disposed between the radially-expandable tube and the frame.

9. The implant device of claim 8, wherein the one or more fluid-filled chambers have foam disposed therein.

10. The implant device of claim 1, wherein the tube is coupled at first and second axial ends thereof to respective first and second axial ends of the frame by at least one of heat-sealing, machine-crimping, or suturing.

11. The implant device of claim 1, wherein the tube is sutured to a suture ring associated with an axial end of the frame.

12. The implant device of claim 1, wherein:
the radially-expandable tube is disposed within a first axial portion of the frame, the axially bulging cylindrical form being associated with the first axial portion; and
the frame includes a second axial portion having a one-way valve associated therewith.

13. The implant device of claim 12, wherein the frame further includes a neck portion disposed between the first axial portion and the second axial portion, the neck portion of the frame having a diameter that is less than a both a maximum diameter of the first axial portion and a maximum diameter of the second axial portion, forming an hourglass shape of the frame.

14. The implant device of claim 13, wherein the second axial portion has a sealing skirt sutured thereto.

15. The implant device of claim 12, further comprising one or more gas-filled chambers disposed between the radially-expandable tube and the frame in the first axial portion of the frame.

16. An implant device for restoring compliance to a blood vessel, the implant device comprising:
a frame having an axially-convex form that expands in diameter moving from first and second axial ends of the frame to a medial portion of the frame; and
a cylindrical toroid balloon member comprising one or more gas-filled chambers disposed at least partially within the frame, the balloon member forming a central axial flow channel.

17. The implant device of claim 16, wherein the one or more gas-filled chambers are formed between an outer layer of the balloon member and an inner layer of the balloon member that forms the central axial flow channel.

18. The implant device of claim 16, wherein the one or more gas-filled chambers occupy a space between the flow channel and the frame.

19. The implant device of claim 16, wherein the one or more gas-filled chambers are configured to radially compress when a pressure within the flow channel is greater than a pressure of gas within the one or more gas-filled chambers.

20. An implant device for restoring compliance to a blood vessel, the implant device comprising:
a tubular frame comprising a first axial segment and a second axial segment;
a radially-expandable tube disposed within the first axial segment of the frame; and
a one-way valve disposed within the second axial segment of the frame.
